(19) United States
(12) Patent Application Publication
(10) Pub. No.: US 2002/0013601 A1
(43) Pub. Date: Jan. 31, 2002

(54) Cavity enlarger method and apparatus

(76) Inventors: Anthony A. Nobles, Fountain Valley, CA (US); Luis J. Maseda, Newport Beach, CA (US)

Correspondence Address: KNOBBE MARTENS OLSON & BEAR LLP, 620 NEWPORT CENTER DRIVE, SIXTEENTH FLOOR, NEWPORT BEACH, CA, 92660, US

(21) Appl. No.: 09/772,397

(22) Filed: Jan. 29, 2001

Related U.S. Application Data
(60) Provisional application No. 60/178,974, filed on Jan. 28, 2000.

(51) Int. Cl. A61M 29/00

(52) U.S. Cl. 606/193

(57) ABSTRACT

A device and method for enlarging and supporting a body cavity are disclosed. One embodiment of the device comprises a tubular, distending balloon having first and second distending members, spaced apart from one another, wherein the distending members are inflatable. A tubular connector interconnects the first and second distending members and forms a conduit which allows for unimpeded passage of objects through the balloon. The balloon is adapted to be inserted into a body cavity in a deflated or semi-deflated state. When the distending members are inflated, an outer surface of the balloon exerts pressure on an interior surface of the body cavity, thereby supporting the body cavity in a distended state while allowing for unimpeded passage of medical instrument and biological material through the balloon.

### CROSS-REFERENCE TO RELATED APPLICATION

This application claims the benefit of U.S. Provisional Application No. 60/178,974, filed Jan. 28, 2000.

### BACKGROUND OF THE INVENTION

1. Field of the Invention

This invention relates generally to medical devices. Specifically, the invention relates to a device and method for enlarging a body cavity. The device may be used, for example, to enlarge a patient's vagina to allow for performing a Pap smear procedure.

2. Description of the Related Art

Currently, it is difficult to enlarge or distend certain organs, vessels, and/or body cavities of a patient without causing discomfort, pain or injury to the patient. For example, using a metallic speculum to enlarge a patient's vagina for a Pap smear procedure often causes discomfort to the patient because the speculum is rigid, cold, and non-conforming to anatomy. In addition, the operator of a speculum often is required to hold the speculum in the patient, thereby making it difficult for the operator to perform additional procedures.

What is needed, therefore, is an improved device and method for enlarging and supporting body cavities that substantially reduces the discomfort and injury to the patient.

### SUMMARY OF THE INVENTION

The present invention relates to a device for enlarging and supporting a body cavity. One embodiment of the device comprises a tubular, distending balloon having first and second distending members, spaced apart from one another, wherein the distending members are inflatable. A tubular connector interconnects the first and second distending members and forms a conduit which allows for unimpeded passage of objects and biological material through the balloon. Another embodiment of the device comprises a tubular, inflatable balloon, having a distal end, a proximal end, at least one central lumen, an outer surface and an inflation tube. The inflation tube is attached to the proximal end of the balloon and is in fluid communication with the balloon. The balloon is adapted to be inserted into a body cavity in a deflated or semi-deflated state. The balloon is further adapted, to be inflated to an inflated state once inserted inside the body cavity. As the balloon is inflated, the outer surface of the balloon expands and distends the body cavity while the central lumen allows for unimpeded passage of objects, such as medical instruments, to pass through the balloon.

In one aspect of the present invention, an expandable device is provided for enlarging a body cavity. The device in its expanded configuration comprises first and second supporting members and a tubular connector having inner and outer surfaces, the connector interconnecting the supporting members. The connector has a first end adjacent the first supporting member and a second end adjacent the second supporting member. The tubular connector has a maximum transverse dimension at its first end less than that of the first supporting member and a maximum transverse dimension at its second end less than that of the second supporting member. The tubular connector has a length greater than the maximum transverse dimension of either the first supporting member or the second supporting member. A lumen is defined by the inner surface of the tubular connector extending through the tubular connector. The tubular connector is adapted to apply force to the body cavity and retract surrounding tissue when the device is in the expanded configuration.

In another aspect of the present invention, the device for enlarging a body cavity comprises an elongate body having inner and outer surfaces extending between a first end of the elongate body and a second end of the elongate body. A longitudinal dimension is generally defined between the first end and the second end with a transverse dimension being perpendicular to the longitudinal dimension. A lumen is defined by the inner surface of the elongate body extending through the elongate body. A first supporting member is connected adjacent the first end of the elongate body, the first supporting member having a maximum transverse dimension that is larger than a maximum transverse dimension of the elongate body at its first end. A second supporting member is connected adjacent the second end of the elongate body, the second supporting member having a maximum transverse dimension that is larger than a maximum transverse dimension of the elongate body at its second end. The elongate body has a length along its longitudinal dimension that is greater than the maximum transverse dimension of either the first supporting member or the second supporting member. The device is expandable between an undeployed position and a deployed position in which the outer surface of the elongate body exerts a force against a wall of the body cavity. An elongate applicator retains the device for insertion into a body cavity, the device arranged on the applicator such that upon deployment the applicator is disposed in the lumen for withdrawal by a user.

In another aspect of the present invention, a method of examining a body cavity is provided. The method comprises inserting an expandable device into the body cavity, the expandable device having a proximal end and a distal end and an inner and outer surface extending between the proximal and distal ends. A lumen is defined by the inner surface extending between the proximal end and the distal end, wherein the longitudinal length between the proximal and distal ends is greater than the maximum transverse dimension of either of the proximal and distal ends, and the outer surface between the proximal and distal ends has a maximum transverse dimension that is less than the maximum transverse dimension of either of the proximal and distal ends. The expandable device is expanded within the body cavity, wherein expansion of the expandable device causes the outer surface between the proximal and distal ends to exert a force against a wall of the body cavity.

In another aspect of the present invention, an apparatus is provided comprising an expandable device having a lumen and an applicator for inserting the expandable device into a body cavity. The applicator comprises a retaining portion which holds at least a portion of the expandable device in a collapsed state while the expandable device is inserted into the body cavity, a handle portion, and shaft portion extending through the lumen between the retaining portion and the handle portion.

In another aspect of the present invention, a method of inserting an expandable device into a body cavity is provided. The expandable device has a proximal end and a distal end and a lumen extending therethrough. The method comprises inserting the expandable device and the applicator into a desired position with the body cavity, the expandable device being at least partially retained within a retaining portion of the applicator. The expandable device is expanded, the applicator is withdrawn through the lumen of the expandable device.

### BRIEF DESCRIPTION OF THE DRAWINGS

FIG. 1 is a perspective view of one embodiment of a device for enlarging body cavities using a distending balloon in accordance with the invention.

FIG. 1A is a perspective view of a light source in an open, deployed state.

FIG. 1B is a perspective view of the light source of FIG. 1A in a wrapped state.

FIG. 2 is a side view of a distending balloon in an inflated state.

FIG. 3A is a partial cross-sectional view of the distending balloon of FIG. 2.

FIG. 3B is a cross-sectional view of the distending balloon of FIG. 2, taken along line 3B-3B of FIG. 3A.

FIG. 3C is a side view of another embodiment of the distending balloon of FIG. 2, wherein a large opening is provided in a tubular connector of the distending balloon.

FIG. 3D is a cut-away view of an embodiment of an expandable cavity enlarger in an expanded configuration.

FIG. 3E is a perspective view of the expandable cavity enlarger of FIG. 3D in a collapsed, narrow configuration.

FIG. 4 generally illustrates the use of the device of FIG. 1 as used in a vagina and in a cervix, wherein large and small distending balloons are shown in an inflated state.

FIG. 4A is a side view of a distending balloon adapted to conform to the anatomy of a cervix.

FIG. 5A is a partial cross-sectional view of another embodiment of the distending balloon of FIG. 2, wherein duckbill valves are provided on a proximal end of the distending balloon.

FIG. 5B is a side view of the proximal end of the distending balloon of FIG. 5A.

FIG. 6 is a side view of another embodiment of a distending balloon in an inflated state.

FIG. 7 is a side view of another embodiment of a distending balloon in an inflated state.

FIG. 8 is a side view of another embodiment of a distending balloon in an inflated state.

FIG. 8A is a side view of another embodiment of a distending balloon in an inflated state.

FIG. 8B is a perspective view of another embodiment of a distending balloon in an inflated state.

FIG. 8C is a perspective view of another embodiment of a distending balloon in an inflated state.

FIG. 9 illustrates another embodiment of a distending balloon in an inflated state.

FIG. 10 is a cross-sectional side view of another embodiment of a distending balloon in an inflated state and enlarging a body cavity.

FIG. 11A illustrates another embodiment of a distending balloon in an inflated state.

FIG. 11B is a cross-sectional view of the distending balloon of FIG. 11A.

FIG. 12 is a cross-sectional view of another embodiment of a distending balloon in an inflated state.

FIG. 13 is a cross-sectional view of another embodiment of a distending balloon in an inflated state.

FIG. 14 is a cross-sectional view of another embodiment of a distending balloon in an inflated state.

FIG. 15 is a side view of one embodiment of a balloon applicator that is used for inserting a distending balloon into a body cavity.

FIG. 16A generally illustrates the use of the balloon applicator of FIG. 15, in which a deflated distending balloon is wrapped onto the balloon applicator and tucked within a retaining hook section of the balloon applicator.

FIG. 16B generally illustrates the withdrawal of the balloon applicator of FIG. 15 through a central lumen of an inflated distending balloon.

FIG. 17 is a perspective view of another embodiment of a balloon applicator that may be used for inserting a distending balloon into a body cavity.

FIG. 17A is a perspective view of another embodiment of a balloon applicator that may be used for inserting a distending balloon into a body cavity.

FIG. 18A generally illustrates the use of the balloon applicator of FIG. 17, wherein a deflated distending balloon is wrapped onto the balloon applicator and partially tucked into a retaining cavity of the balloon applicator.

FIG. 18B generally illustrates the withdrawal of the balloon applicator of FIG. 17 through a central lumen of an inflated distending balloon.

FIG. 18C is a perspective view of another embodiment of a balloon applicator that is used for inserting a distending balloon into a body cavity.

FIG. 19 is a perspective view of another embodiment of a balloon applicator that may be used for inserting a distending balloon into a body cavity.

FIG. 20A generally illustrates the use of the balloon applicator of FIG. 19, in which a distending balloon is deflated and inserted into a retaining cavity of the balloon applicator.

FIG. 20B generally illustrates the withdrawal of the balloon applicator of FIG. 19 through a central lumen of an inflated distending balloon.

FIG. 21 is a perspective view of a mandrel that is used to form a balloon member.

FIG. 22 is a side view of a mandrel that may be used to form a single, continuous one-piece balloon member, with a balloon member shown thereon in cross-section.

FIG. 23A is a cross-sectional side view of a single, continuous one-piece balloon member formed using the mandrel of FIG. 22, with the enclosed end trimmed to create an opening.

FIG. 23B is a cut away view illustrating how the balloon member of FIG. 22 is folded into itself to create the device in accordance with one embodiment of the invention.

### DETAILED DESCRIPTION OF THE PREFERRED EMBODIMENTS

The preferred embodiments of the present invention comprise a cavity enlarger adapted to enlarge, expand or support a body cavity of a patient, such as a vagina, a rectum, a urethra, a fallopian tube, an esophagus, etc. The length, diameter, and size of the apparatus are selected to conform to the anatomy of the surrounding tissue of the particular organ, lumen or body cavity. In accordance with one embodiment of the present invention, a device for enlarging a body cavity using a distending balloon is described herein. It will be appreciated that this invention should not be limited to embodiments using balloons, and thus, other embodiments, including those which employ other types of expandable devices, are also contemplated. In order to fully specify the preferred design, various embodiment specific details are set forth. It should be understood, however, that these details are provided only to illustrate the preferred embodiments, and are not intended to limit the scope of the present invention.

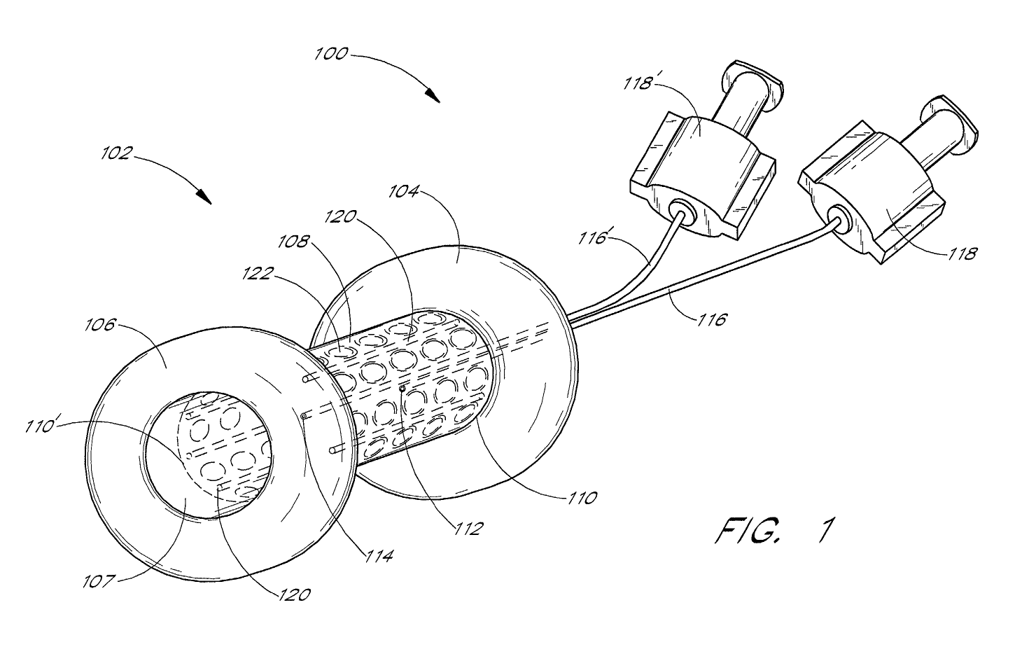

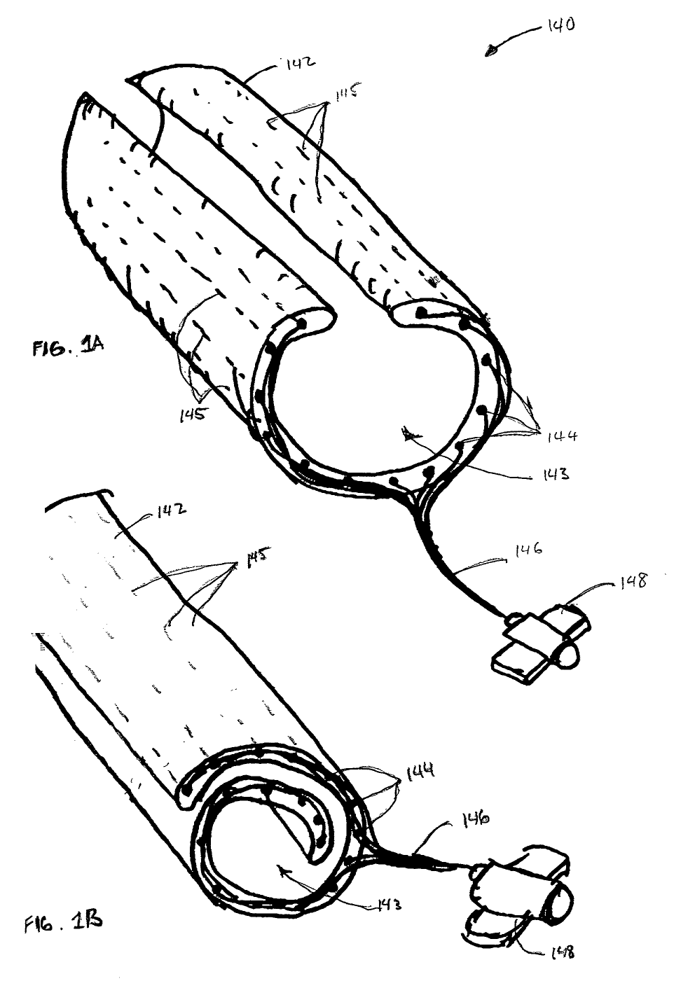

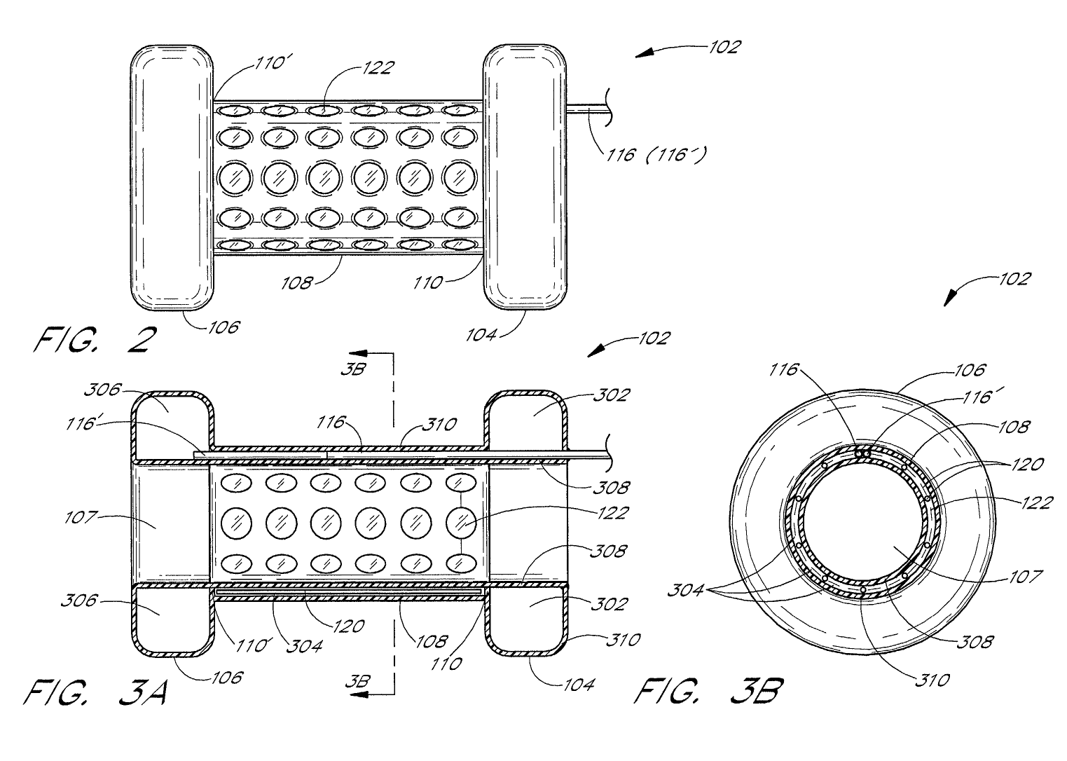

With reference to FIG. 1, a preferred embodiment of the invention provides a device 100 for enlarging body cavities using a distending balloon 102. The balloon 102 comprises first and second supporting members, which are more preferably first and second distending members 104, 106, a tubular connector 108, a central lumen 107, a plurality of support ribs 120, and a plurality of supportive depressions 122. The term “tubular” is used herein with reference to an object having an interior cavity that spans substantially the length of the object, and is not limited to objects of circular crosssection or to interior cavities of circular cross-section. It will be appreciated that many different interior and exterior cross-sectional shapes and sizes may be utilized, such as, by way of example, triangular, diamond-shaped, square-shaped, etc. It will be further appreciated that different cross-sectional shapes may advantageously be combined, thereby forming additional cross-sectional shapes. In the embodiment illustrated in FIGS. 1 and 2, the tubular connector 108 interconnects the first and second distending members 104, 106. The distending members 104, 106 and the tubular connector 108 are preferably made of a single, continuous one-piece balloon member that provides at least one inflatable chamber. In the preferred embodiment, the distending members 104, 106 and the tubular connector 108 provide three interior chambers, which will be discussed in more detail below.

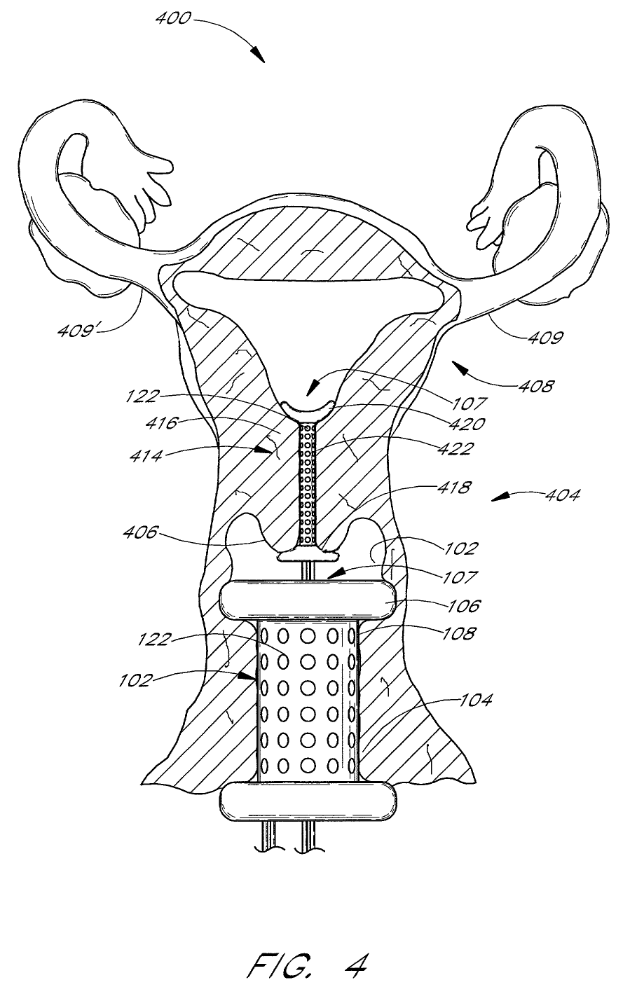

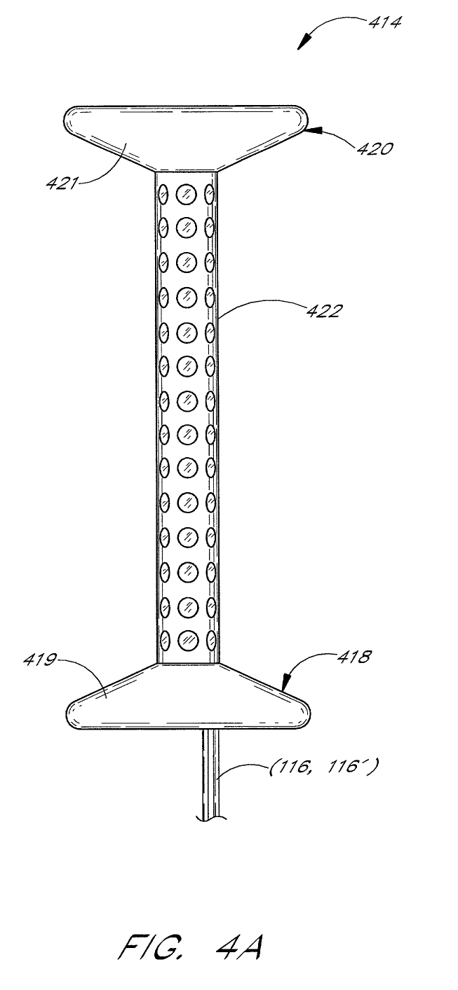

In the embodiment illustrated in FIG. 1, the distending balloon 102 has a length that is greater than a diameter of the distending members 104, 106. In another embodiment, the length of the balloon 102 may advantageously be equal to the diameter of the distending member 104, 106. In still another embodiment, the length of the balloon 102 may advantageously be smaller that the diameter of the distending members 104, 106. Furthermore, each of the distending members 104, 106 has a width that is smaller than a diameter of the tubular connector 108. In other embodiments, the width of the distending members 104, 106 may be equal to or greater than the diameter of the tubular connector 108. The tubular connector 108 and the distending members 104, 106 may be of any geometrical cross-section, ranging from three vertices (i.e., triangular) to a multiple-vertices shape, such as circular. In one embodiment, for use with a vagina 404 (FIG. 4), the distending balloon 102 has an overall length ranging from about 8 centimeters to about 12 centimeters, and a tubular connector 108 having an outer diameter ranging from about 5 to 8 cm. Those of ordinary skill in the art will realize that the relative dimensions of the balloon 102, the distending members 104, 106, and the tubular connector 108 may be determined based on a particular medical procedure contemplated, and as such may be substantially changed without detracting from the invention.

The distending balloon 102 is preferably made of flexible, semi-compliant material. The term “semi-compliant” is used herein in reference to a material that is sufficiently non-compliant to prevent the balloon 102 from over-expanding when inflated to an optimal inflated state. The material is also flexible to allow the balloon 102 to be bent and inserted into various regions of a patient's body. In one embodiment, the balloon 102 is made of polyurethane. In another embodiment, the balloon 102 may be made of polypropylene. In still another embodiment, the balloon 102 may be made of silicone. Other embodiments include other non-compliant or semi-compliant materials, or blends thereof, including but not limited to EVA (Ethylene-Vinyl-Acetate), PVC, PET, and NYLON. Those of ordinary skill in the art will recognize that the balloon 102 may advantageously be made of other non-compliant or semi-compliant, biocompatible materials without detracting from the invention.

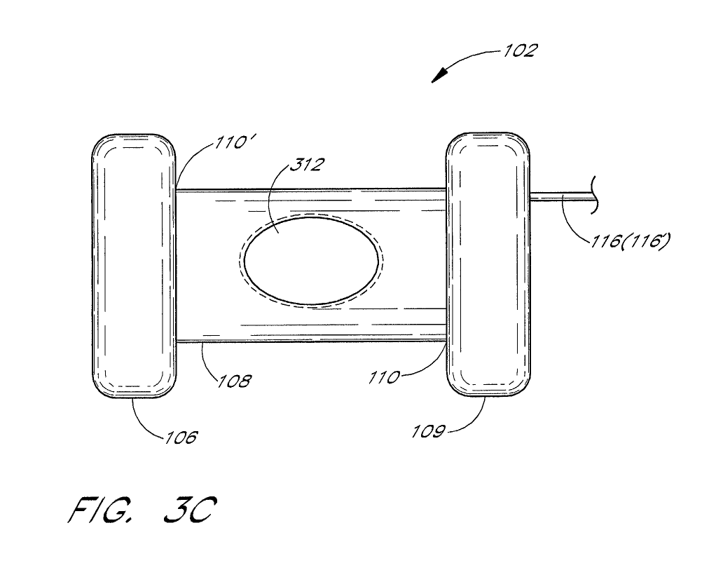

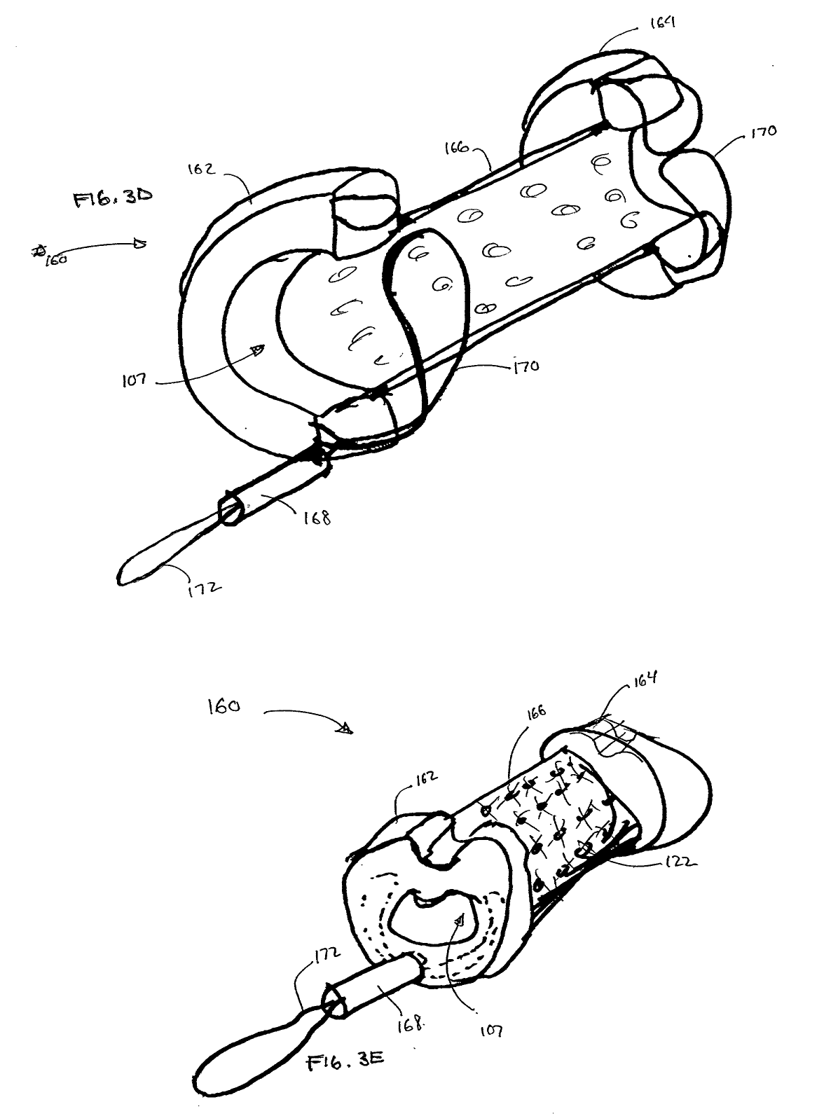

As illustrated in FIGS. 1 and 2, a first annular seal 110 is formed between the first distending member 104 and the tubular connector 108. Similarly, a second annular seal 110′ is formed between the tubular connector 108 and the second distending member 106. The annular seals 110, 110′ are formed circumferentially between inner and outer layers 308, 310 (FIGS. 3A and 3B) of the balloon 102, using radio frequency (RF) welding, ultrasound welding, thermal bonding, adhesive, or other suitable sealing techniques.

Referring to FIG. 3A, the annular seals 110, 110′ form three distinct chambers within the balloon 102: a first inflation chamber 302, a central inflation chamber 304, and a second inflation chamber 306. The first inflation chamber 302 is an interior cavity of the first distending member 104, formed by the annular seal 110. The central inflation chamber 304 is an interior cavity of the tubular connector 108, and is formed by the annular seals 110, 110′. The second inflation chamber 306 is an interior cavity of the second distending member 106, formed by the annular seal 110′. In the illustrated embodiment, the annular seal 110 preferably includes a duct or unsealed passage that allows for fluid communication between the first and central inflation chambers 302, 304, as described below, to allow the first inflation chamber 302 and the central inflation chamber 304 to be inflated together.

In another embodiment, the tubular connector 108 may be a separate component, which interconnects the first and second distending members 104, 106. In addition, the balloon 102 can alternatively be provided with several internal chambers that are separately inflatable. For example, the balloon 102 can be constructed such that the first, second, and central inflation chambers 302, 306, 304 (FIG. 3A and 3B) are separate and independent chambers. In this embodiment, the first annular seal 110 made at the junction between the first distending member 104 and the tubular connector 108, and the second annular seal 110′ formed at the junction between the second distending member 106 and the tubular connector 108, completely seal off their respective chambers. As discussed with reference to FIG. 3A, the annular seals 110, 110′ can be formed circumferentially between inner and out layers 308, 310 (FIGS. 3A and 3B) of the balloon 102, using radio frequency (RF) welding, ultrasound welding, thermal bonding, adhesive, or other suitable sealing techniques.

Referring to FIGS. 1, 3A and 3B, the tubular connector 108 preferably comprises the inner and outer layers 308, 310 of the balloon 102, the support ribs 120, and the supportive depressions 122. As illustrated in FIGS. 3A and 3B, the support ribs 120 are placed within the central inflation chamber 304 between the inner and outer layers 308, 310 of the balloon 102. The support ribs 120 are preferably uniformly distributed around the circumference of the central inflation chamber 304 and are parallel to the tubular connector 108. Furthermore, the support ribs 120 are held in position by the supportive depressions 122 and the annular seals 110, 110′. The support ribs 120 may be made of plastic, metal, or some other rigid material. The support ribs 120 and the supportive depressions 122 maintain the tubular connector 108 in an essentially cylindrical configuration when the balloon 102 is inflated and used to support a body cavity.

In another embodiment, the support ribs 120 may be positioned transversely or diagonally relative to the tubular connector 108. In still another embodiment, the support ribs 120 may be positioned relative to the tubular connector 108 such that the support ribs 120 form a weave or other pattern within the central inflation chamber 304. In other embodiments, the support ribs 120 may comprise additional material which intrudes or protrudes from the tubular connector 108, thereby increasing the structural strength and/or rigidity of the tubular connector 108. Those of ordinary skill in the art will realize that the relative orientations of the support ribs 120 and the tubular connector 108 may be substantially changed without detracting from the invention.

In a preferred embodiment, the supportive depressions 122 are localized regions of the tubular connector 108 in which the inner and outer layers 308, 310 of the balloon 102 are adhered or bonded together. In another embodiment, the supportive depressions 122 may be holes which allow medical instruments, such as an endoscope, to pass unimpeded through the inner and outer layers 308, 310 of the tubular connector 108. In still another embodiment, the supportive depressions 122 may be openings that are substantially larger in size than illustrated in FIGS. 1 and 2. In yet another embodiment, the supportive depressions 122 may be composed of transparent material, thereby forming “windows” in the tubular connector 108. Such windows may advantageously facilitate visual inspection of body cavities. In addition, the shape of the windows may advantageously be changed based on the type of medical procedure contemplated. In the preferred embodiment, the supportive depressions 122 are formed by using radio frequency (RF) welding, ultrasound welding, thermal bonding, adhesive, or other suitable bonding techniques.

Alternatively, openings may advantageously be formed in the tubular connector 108. These openings are preferably either open or formed of a transparent material. In one embodiment, illustrated in FIG. 3C, the tubular connector 108 comprises one large opening 312 which allows for unimpeded passage of medical instruments and biological material through the inner and outer layers 308, 310 of the tubular connector 108. In another embodiment, a plurality of openings 312 of varying sizes may advantageously be formed on the tubular connector 108 in varying radial, helical, or longitudinal patterns. In still another embodiment, the openings 312 may advantageously be filled with a transparent material, thereby forming windows which facilitate visual inspection of interior surfaces of body cavities. In the illustrated embodiment of FIG. 3C, it is contemplated that the distending members 104, 106 may be inflated with or without inflating the tubular connector 108.

In another embodiment, the distending balloon 102 may be made of a transparent material to facilitate visual inspection of body cavities and/or transmission of light therein. In one embodiment, specific segments or sections of the balloon 102 may be made of transparent material. For example, the tubular connector 108 may be made of a single layer of transparent material while the distending members 104, 106 are made of a translucent material. In another embodiment, the entirety of the balloon 102 may be made of transparent or translucent material. A person skilled in the art will realize that the opacity of the balloon 102, or individual portions thereof, may be substantially altered without detracting from the invention.

In another embodiment, the tubular connector 108 may comprise a single layer of transparent material with an embedded or attached light source, such as by way of example, a fiber-optic array, LED, or similar light source. It is contemplated that any type of light may be used, such as, by way of example, Ultraviolet (UV) light, Infrared (IR) light, or visible light. The light source may advantageously be used for illumination of body cavities and/or medical procedures involving an application of light to tissue, such as drug activation, light therapy on tissue, and the like. With this embodiment, the tubular connector 108 is non-inflatable, the supportive force being provided entirely by the distending members 104, 106. In another embodiment, portions of the tubular connector 108, and/or the distending members 104, 106, may be made of an opaque material in order to isolate light emission within body cavities. In still another embodiment, portions of the tubular connector 108, and/or the distending members 104, 106 are made of an opaque material, formed such that light may be localized with body cavities. In yet another embodiment, the central lumen 107 may advantageously be filled with liquid media in order to aid light diffusion within body cavities. A person of ordinary skill in the art will recognize that the type of light source used, and the method of coupling the light source with the distending balloon 102, may be substantially changed without detracting from the invention.

FIGS. 1A and 1B illustrate one embodiment of a light source 140 that may be used with the distending balloon 102. FIG. 1A shows the light source 140 in an open or deployed state. FIG. 1B shows the light source 140 is a narrow, wrapped state. The light source 140 comprises a C-shaped sleeve 142, a central lumen 143, a fiberoptic array 145, a fiber-optic cable 146, and a fiber-optic light connector 148. The fiber-optic array 145 further comprises a plurality of fiber-optic lines 144. The fiberoptic lines 144 are preferably embedded within the material comprising the C-shaped sleeve 142. In another embodiment, the fiber-optic lines 144 may be attached to the interior and/or exterior of the C-shaped sleeve 145. The C-shaped sleeve 142 is made of a flexible, transparent or translucent material to allow light transmission through the C-shaped sleeve 142. As illustrated in FIG. 1A, the fiber-optic lines 144 protrude from the proximal end of the C-shaped sleeve 142, and are bundled together, thereby forming the fiber-optic cable 146. The fiber-optic cable 146 is then attached to the fiber-optic light connector 148.

In operation, an operator preferably places the C-shaped sleeve 142 into the narrow, wrapped state illustrated in FIG. 1B. The light source 140 may be utilized either outside or inside of the distending balloon 102. When the light source 140 is used on the outside of the distending balloon 102, the C-shaped sleeve 142 may be wrapped around an exterior surface of the tubular connector 108. When the light source 140 is used on the inside of the distending balloon 102, the C-shaped sleeve 142 may be placed within the central lumen 107 of the distending balloon 102, coincident with an interior surface of the tubular connector 108.

When the fiber-optic light connector 148 is attached to a source of light, the fiber-optic cable 146 transmits light to the fiber-optic array 154 via the fiber-optic lines 144. The fiber-optic array 145 illuminates the central lumen 143 of the C-shaped sleeve 142. Such illumination may advantageously be used for illumination of body cavities and/or medical procedures involving an application of light to tissue, such drug activation, light therapy on tissue, and other similar procedures.

Referring again to FIG. 1, first and second inflation tubes 116, 116′ are coupled to the balloon 102. In the illustrated embodiment of FIG. 1, it is contemplated that the first and second inflation tubes 116, 116′ each have at least one internal lumen. Within the first inflation tube 116 is an inflation lumen 112 which opens into the central inflation chamber 304 (FIGS. 3A and 3B) and is used to inflate both the first distending member 104 and the tubular connector 108, through the opening in the annular seal 110. Within the second inflation tube 116′ is an inflation lumen 114 which opens into the second inflation chamber 306 and is used to inflate the second distending member 106. A standard luer connector 118, which is adapted to receive a syringe (not shown), provides access to the inflation lumen 112. Similarly, a luer connector 118′, which is adapted to receive a syringe, provides access to the inflation lumen 114. Using the syringes, the balloon 102 (including the distending members and the tubular connector 104, 106, 108) can be inflated with an appropriate fluid such as air, water, or saline solution.

It will be recognized that the first and second inflation tubes 116, 116′ can accommodate additional inflation lumens (not shown). For example, in one embodiment, additional lumens may be utilized such that the first distending member 104, the second distending member 106, and the tubular connector 108 can be inflated independently of each other when the chambers of each member are sealed against fluid communication. In another embodiment, independent inflation of the distending members 104, 106 and the tubular connector 108 may advantageously be achieved by employing a third inflation tube (not shown). Those of ordinary skill in the art will recognize that the number of inflation tubes, as well as the numbers of lumens incorporated therein, may be varied without detracting from the invention.

Alternatively, the balloon 102 can be constructed such that the distending members 104, 106 can be inflated without inflating the tubular connector 108. Specifically, the first annular seal 110 can be formed at the junction between the first distending member 104 and the tubular connector 108, and the second annular seal 110′ can be formed at the junction between the second distending member 106 and the tubular connector 108. The seals 110, 110′ are formed between the inner and outer layers 308, 310 (FIGS. 3A and 3B) of the balloon 102 such that fluid is prevented from entering the tubular connector 108.

As another alternative, the supporting members 104 and 106 are not necessarily distending members, but in one embodiment, may be made of solid pieces such as rubber. In another embodiment, balloon 102 can be constructed such that the distending members 104, 106 are not inflated, but rather are mechanically expandable. As illustrated in FIG. 3D, one embodiment of a cavity enlarger 160 comprises first and second distending members 162, 164, a tubular connector 166, a central lumen 107, support wires 170, a distal support wire 172, and a guide tube 168. The construction of the tubular connector 166 is substantially similar to the construction of the tubular connector 108, discussed with reference to FIGS. 1 through 3B, except that the tubular connector 166 in this embodiment is non-inflatable. In another embodiment, the tubular connector 166 may be of a single layer construction. The distending members 162, 164 are solid annuli made of a flexible, biocompatible material, each embedded with a support wire 170. The support wires 170 are coupled together, and are operatively coupled to the distal support wire 172. In one embodiment, the support wires 170 and the distal support wire 172 comprise one segment of wire. In another embodiment, the support wires 170 and the distal support wire 172 are separate segments of wire that are attached to each other during assembly of the cavity enlarger 160. The support wires 170 and the distal support wire 172 may be made of any substantially rigid material capable of passing from an expanded ring configuration to a collapsed, narrow configuration. The support wires 170 and the distal support wire 172 are preferably made of a Shape Memory Alloy (SMA).

During operation of the cavity enlarger 160, an operator preferably pulls on the distal support wire 172 to move the support wires 170 from the expanded ring configuration to the collapsed, narrow configuration. This causes the first and second distending members 162, 164 to collapse, as illustrated in FIG. 3E. As the distending members 162, 164 collapse, the cavity enlarger 160 is folded onto itself, thereby assuming a narrow configuration. The operator then inserts the cavity enlarger 160 into a body cavity of a patient. Once the cavity enlarger 160 is positioned within the body cavity the operator releases the distal support wire 172, allowing the support wires 170 to pass from the collapsed, narrow configuration to the expanded ring configuration. This causes the first and second distending members 162, 164 to expand, thereby expanding the tubular connector 166. As the tubular connector 166 expands, it distends and supports the body cavity.

It will be appreciated that other types of expansion mechanisms, for both the supporting members 162 and 164, as well as for the tubular connector 166, are also contemplated as falling within the scope of this invention.

Referring again to the preferred embodiment of FIGS. 1 through 3B, the inflation lumens 112, 114 may serve an additional purpose of preventing an over-inflation of the balloon 102. In one embodiment, an over-inflation balloon (not shown) is attached to the proximal ends of the inflation lumens 112, 114. Each over-inflation balloon is attached to a luer connector that is attached to a luer fitting. A one-way, syringe-activated valve is built inside each luer connector. Each over-inflation balloon provides a space for sliding the distal part of the corresponding valve. In a preferred embodiment, the over-inflation balloons are ‘Pilot’ balloons made by Mallinckrodt Medical, Inc. When a physician inserts syringes into the luer fittings, and the corresponding valves, to inflate the balloon 102, a component inside each valve moves distally to allow the syringes to inject the inflation fluid. If the physician removes the inflation syringes from the valves, the valves close (the component inside each valve moves proximally) and prevent the balloon 102 from losing inflation. To deflate the balloon 102, the physician inserts the syringes into the valves and withdraws the fluid.

When the balloon 102 begins to inflate, there is no resistance on the balloon 102 as it expands. Consequently, there is no backpressure in the inflation lumens 112, 114. However, when the balloon 102 inflates to a predetermined diameter, or nears a maximum diameter, backpressure builds up in the inflation lumens 112, 114, and the over-inflation check balloons begin to inflate and bulge. This provides a direct signal to the physician that the inflated balloon 102 has expanded to the predetermined diameter. The threshold pressure-level needed to inflate the over-inflation balloons may also be produced by attempts to inflate the balloon 102 beyond its maximum diameter, even though the balloon 102 may not be in contact with a body cavity.

Alternatively, in addition to the over-inflation balloons, some other pressure-indicating device, such as a pressure meter, may be used to indicate that a desired pressure level has been reached within the balloon 102. Such a pressure-indicating device may be fluidly coupled to the balloon 102. In another embodiment, the over-inflation check balloons or other pressure-indicating devices may be coupled to separate lumens (not shown) which run parallel with the inflation lumens 112, 114, along the inflation tubes 116, 116′, and extend to an opening coinciding in position with the interior chambers of the balloon 102. Those of ordinary skill in the art will realize that in other embodiments additional lumens and luer connectors may advantageously be provided, whereby additional functions may be performed.

FIG. 4 generally illustrates the function of the distending balloon 102 as used in a female reproductive system 400. It is to be understood, however, that the balloon 102 may be utilized for performing a wide variety of other medical procedures, such as by way of example, laparoscopic procedures performed for diagnostic or surgical purposes. As illustrated in FIG. 4, the female reproductive system comprises a vagina 404, a cervix 406, a uterus 408, and Fallopian tubes 409, 409.′ It is contemplated that the balloon 102, depicted in FIG. 4, is designed such that it conforms to the anatomy of the vagina 404. In one embodiment, the tubular connector 108 has an outer diameter ranging up to about 5 centimeters. In operation, a physician places the balloon 102 in a deflated or semi-deflated state and then inserts the balloon 102 into a patient's vagina 404. The physician may use a balloon applicator to insert the balloon 102, discussed in greater detail below.

Once the balloon 102 is placed in a desired position, the physician inflates the balloon 102 via inflation tubes 116, 116′ with saline solution, water, air, or other suitable fluid. While the balloon 102 inflates, the distending members 104, 106 expand, thereby opening the tubular connector 108. As the tubular connector 108 opens it exerts a pressure on an inner surface 402 of the vagina 404. As the balloon 102 is further inflated, the tubular connector 108 opens and supports the vagina 404 in a distended state. While the inflated balloon 102 supports the vagina 404, the distending members 104, 106 hold the balloon 102 in place, thereby minimizing the movement of the balloon 102 relative to the vagina 404. Further, the distending members 104, 106 extend radially outward beyond the tubular connector 108 such that the distending members 104, 106 provide most, or nearly all, of the force against the inner surface 402 via the expansion of the tubular connector 108. This serves to maintain an essentially cylindrical configuration of the tubular connector 108 while the balloon 102 is being used to support the vagina 404. The support ribs 120 (FIGS. 1, 3A, and 3B) and supportive depressions 122 provide additional support to the tubular connector 108.

When the balloon 102 reaches an optimal inflated state, as shown in FIG. 4, the physician ceases inflation of the balloon 102. In a preferred embodiment, the physician inflates the balloon 102 with a predetermined volume of fluid, which properly inflates the balloon 102 to the optimal inflated state. With this embodiment, the volume of fluid required to optimally inflate the balloon 102 is measured beforehand, thereby facilitating proper inflation of the balloon 102 when it is used to support a body cavity. In another embodiment, the physician may use pressure-indicating devices (not shown) coupled to the inflation tubes 116, 116′ to determine when the balloon 102 reaches the optimal inflated state.

With the balloon 102 in the optimal inflated state, the central lumen 107 provides for direct visual examination of the vagina 404 and the cervix 406. Furthermore, medical instruments, such as an endoscope, or biological material may pass from one end of the balloon 102 through the central lumen 107 to the other end of the balloon 102. Thus, the central lumen 107 provides direct access to the cervix 406, the uterus 408, and the Fallopian tubes 409, 409′ while the balloon 102 supports the vagina 404. The physician may perform a vaginal/cervical examination, or pass instruments through the central lumen 107 to perform a medical procedure, such as tissue sampling or a Pap smear.

Before removing the balloon 102 from the patient's vagina 404, the physician may withdraw inflation fluid from the first and central inflation chambers 302, 304, thereby placing the first distending member 104 and the tubular connector 108 is a deflated or semi-deflated state while leaving the second distending member 106 in the inflated state. The physician can then use a finger to move the proximal portion of the tubular connector 108 away from the inner surface 402 of the vagina 404 and then conduct a visual examination of the vaginal wall. Furthermore, the physician may leave the second distending member 106 in the inflated or semi-inflated state while withdrawing the balloon 102 from the vagina 404. With this procedure, the physician looks through the central lumen 107 of the balloon 102 and visually observes the response of the vaginal wall as the second distending member 106 passes over the inner surface 402.

Additionally, medical procedures involving the uterus 408 and the Fallopian tubes 409, 409′ are contemplated. In one embodiment, with or without the balloon 102 supporting the vagina 404, as illustrated in FIG. 4, the operator preferably uses a small distending balloon 414 to enlarge and support the cervix 406 in a distended state, thereby gaining direct access to the interior of the uterus 408 and the Fallopian tubes 409, 409′. As seen in FIG. 4A, the small distending balloon 414 is substantially similar in construction to that of the balloon 102, with the exception that the small balloon 414 is of a reduced size and is designed such that it conforms to the anatomy of the cervix 406. The small balloon 414 comprises first and second distending members 418, 420, spaced apart and interconnected by a tubular connector 422. The first distending member 418 has a distal section 419 that conforms to the anatomy of the proximal opening of the cervix 406. In one embodiment, the first distending member 418 folds over the tubular connector 422 to conform to the shape of the cervix. Similarly, the second distending member 420 has a proximal section 421 that conforms to the anatomy of the distal opening of the cervix 406. The tubular connector 422 has a construction that is substantially similar to the construction of the tubular connector 108, with the exception that the tubular connector 422 is preferably smaller. In one embodiment, the tubular connector 422 has an outer diameter preferably ranging from about 0.03 centimeters to 3 centimeters.

Referring again to FIG. 4, the procedure for inserting the small balloon 414 into the cervix 406 is substantially similar to the procedure, discussed above, for inserting the distending balloon 102 into the vagina 404. The operator passes the small balloon 414, in a semi-deflated or deflated state, through the central lumen 107 of the distending balloon 102 and then inserts the small balloon 414 into the cervix 406. The operator then inflates the small balloon 414 with saline solution, water, or other suitable fluid. When the small balloon 414 inflates, the distending members 418, 420 expand, thereby opening the tubular connector 422. As the tubular connector 422 opens it exerts a pressure on an inner surface 416 of the cervix 406. As the balloon 414 inflates further, the tubular connector 420 opens and supports the cervix 406 in a distended state.

While the inflated small balloon 414 supports the cervix 406, the distending members 418, 420 hold the balloon 414 in position, thereby minimizing movement of the balloon 414 relative to the cervix 406. In addition, the support ribs 120 (FIGS. 1, 3A, and 3B) and the supportive depressions 122 provide support to the tubular connector 422, thereby maintaining the cylindrical configuration of the tubular connector 422 when the small balloon 414 is used to support the cervix 406.

Once the small balloon 414 is inflated to an optimal inflated state, the central lumen 107 provides for direct visual examination of the cervix 406 and the uterus 408, and allows for unimpeded passage of material and objects through the balloon 414 while the balloon 414 supports the cervix 406. The operator may pass instruments through the central lumen 107 to perform medical procedures involving the uterus 408 and/or the Fallopian tubes 409, 409′. When the operator finishes performing medical procedures, the operator withdraws the inflation fluid from the small balloon 414, thereby placing the balloon 414 in a deflated or semi-deflated state. The physician then withdraws the balloon 414 from the cervix 406 through the central lumen 107 of the balloon 102.

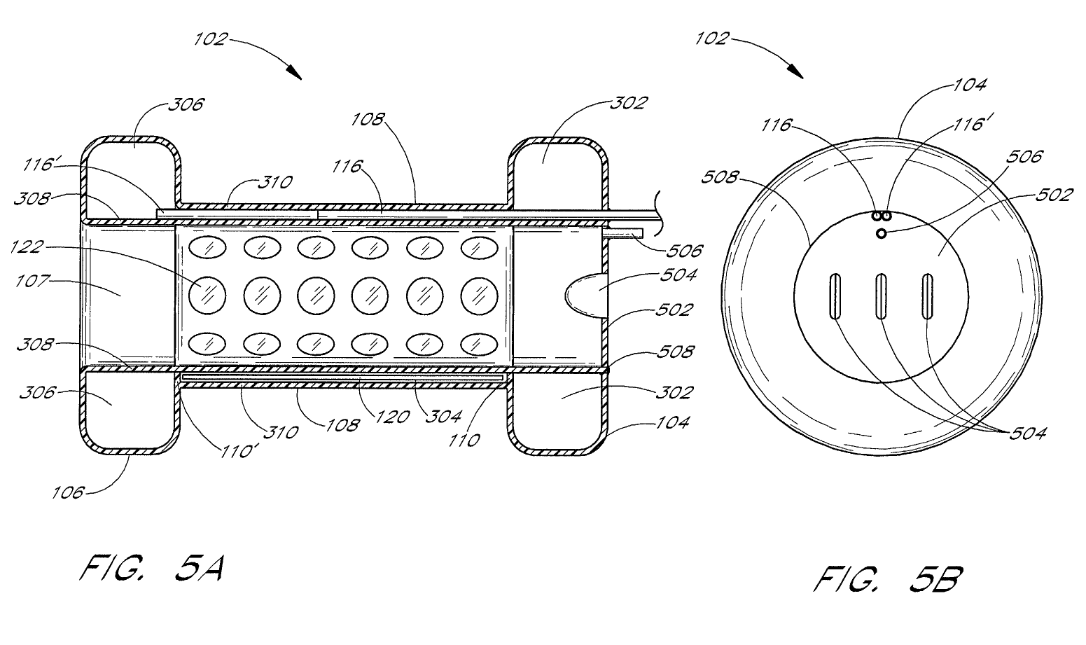

FIGS. 5A and 5B illustrate another embodiment of the distending balloon 102 in an inflated state. The structure of the distending balloon 102 of FIGS. 5A and 5B is substantially similar to the structure of the balloon 102 illustrated in FIGS. 1 through 3A, with the exception of a proximal end surface 502, a plurality of valves 504, a duct 506, and an annular seal 508. As shown in FIG. 5A, the proximal end surface 502 is adhered to the first distending member 104 such that the proximal opening of the central lumen 107 is closed. The annular seal 508 is formed at the junction between the first distending member 104 and the proximal end surface 502. The annular seal 508 is formed by using radio frequency (RF) welding, ultrasound welding, thermal bonding, adhesive, or other suitable sealing techniques.

At least one valve 504, more preferably a duckbill valve, is affixed to the proximal end surface 502. In the embodiment illustrated in FIG. 5B, three duckbill valves 504 are provided. The duckbill valves 504 allow medical devices, such as endoscopic or tissue sampling instruments, to pass through the proximal end surface 502 and the central lumen 107 while preventing fluids, such as blood or other biological matter, from flowing out of the central lumen 107.

The proximal end surface 502 further includes the duct 506. The duct 506 allows fluid to pass through the proximal end surface 502 to or from the central lumen 107 of the balloon 102. In one embodiment, the duct 506 is open-ended tube which facilitates the transfer of fluid, such as saline solution, water, or air, to or from the central lumen 107. In another embodiment, the duct 506 may advantageously include a one-way valve that facilitates the injection of fluid into the central lumen 107 of the balloon 102 while preventing the fluid from flowing out of the central lumen 107 when the injection process is ceased. The operator may advantageously inject a predetermined volume of fluid through the duct 506, thereby filling the central lumen 107 and the body cavity under examination with an optimal volume of fluid. In still another embodiment, a pressure-indicating device (not shown) may advantageously be coupled to the duct 506 to indicate to the physician when the injected fluid has reached an optimal pressure.

In operation, the physician places the balloon 102, illustrated in FIGS. 5A and 5B, into a deflated or semi-deflated state and then inserts the balloon 102 into a body cavity, such as a patient's vagina 404. Next, the physician inflates the balloon 102 according to the procedure discussed with reference to FIG. 4. Once the balloon 102 is sufficiently inflated, the physician injects a fluid, such as saline solution, water, or other suitable fluid, into the duct 506, thereby filling the central lumen 107 of the balloon 102 and the body cavity under examination. In the application where the balloon 102 is used to distend a patient's vagina 404, the fluid injected through the duct 506 fills the central lumen 107 and the vagina 404. Next, the physician inserts a medical instrument, such as an endoscope, into one of the duckbill valves 504 and then advances the instrument through the central lumen 107 of the balloon 102 to a desired location within the vagina 404, such as the cervix 406. The duckbill valve 504 forms a fluid-tight seal around the medical instrument, thereby preventing fluid from flowing out of the central lumen 107 of the balloon 102.

Once the medical procedure is completed, the physician withdraws the medical instrument out of the central lumen 107 through the duckbill valve 504. The physician then withdraws the fluid from the patient and the central lumen 107 of the balloon 102 through the duct 506. Next, the physician deflates and withdraws the balloon 102 from the patient.

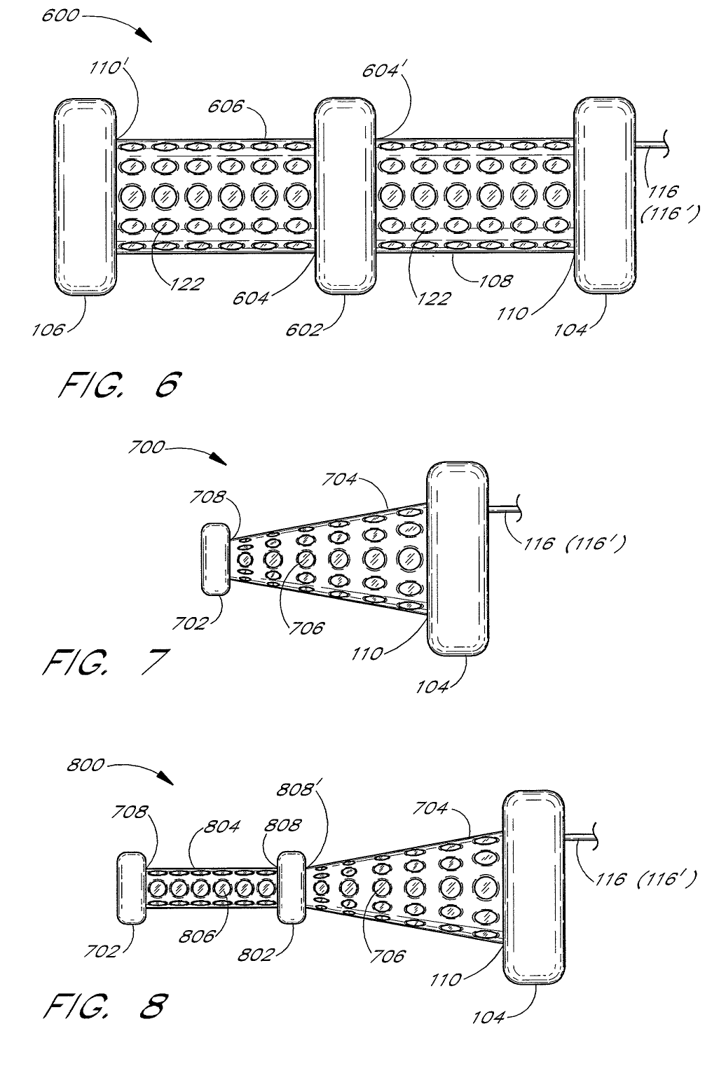

FIG. 6 illustrates another embodiment of a distending balloon 600 in an inflated state. As can be seen, the balloon 600 is substantially similar to the distending balloon 102 of FIG. 2, with the exception of an auxiliary distending member 602 and an auxiliary tubular connector 606. The tubular connector 108 interconnects the first and auxiliary distending members 104, 602, and the auxiliary tubular connector 606 interconnects the auxiliary and second distending members 602, 106. In the illustrated embodiment, it is contemplated that the distending members 104, 602, 106 and the tubular connectors 108, 606 are made of a single, continuous one-piece balloon member that provides at least one internal inflatable chamber. An annular seal 604 is formed between the auxiliary distending member 602 and the auxiliary tubular connector 606, and an annular seal 604′ is formed between the tubular connector 108 and the auxiliary distending member 602. The annular seal 110 is formed between the tubular connector 108 and the first distending member 104, and the annular seal 110′ is formed between the auxiliary tubular connector 606 and the second distending member 106. The annular seals 110, 110′, 604, 604′ are formed circumferentially between inner and outer layers (not shown) of the balloon 600 using radio frequency (RF) welding, ultrasound welding, thermal bonding, adhesive, or other suitable sealing techniques. When these seals completely connect the inner and outer layers of the balloon 600, five separate chambers are formed within the balloon 600.

In the illustrated embodiment, it is contemplated that the construction of the auxiliary tubular connector 606 is substantially similar to that of the tubular connector 108 (FIGS. 3A and 3B). The tubular connector 606 comprises inner and outer layers of the balloon 600, wherebetween a plurality of support ribs 120 (such as illustrated above in FIGS. 1 and 3B) are distributed uniformly around the circumference of the auxiliary tubular connector 606, and oriented parallel to the auxiliary tubular connector 606. The support ribs 120 are held in position by the supportive depressions 122 and the annular seals 604, 110′. The support ribs 120 and the supportive depressions 122 maintain the inflated configuration of the tubular connector 606 when the balloon 600 is used to support a body cavity. In addition, the supportive depressions 122 may be altered such that holes, openings, and/or windows are incorporated into the tubular connector 108 as discussed with reference to FIGS. 1 through 3B.

Referring again to FIG. 6, the first and second inflation tubes 116, 116′ are coupled to the balloon 600, as discussed above with reference to FIG. 1. In the illustrated embodiment of FIG. 6, it is contemplated that the first inflation tube 116 is used to inflate the first distending member 104 and the tubular connector 108, and that the second inflation tube 116′ is used to inflate the auxiliary distending member 602, the auxiliary tubular connector 606, and the second distending member 106. Thus, in this embodiment, the seals 110, 604, and 110′ each has an opening to allow fluid communication between adjacent chambers. It will be recognized that the first and second inflation tubes 116, 116′, as well as any additional inflation tubes that may be optionally included, can each accommodate a plurality of inflation lumens (not shown). As an example, additional lumens and/or inflation tubes may advantageously be utilized such that the distending members 104, 106, 602 and the tubular connectors 108, 606 can be inflated independently of each other when each of the seals between the adjacent chambers is completely closed. Those of ordinary skill in the art will realize that the quantity of inflation tubes and the number of lumens therein may advantageously be changed without detracting from the invention.

In another embodiment, the balloon 600 may advantageously be constructed such that the distending members 104, 106, 602 can be inflated without inflating the tubular connectors 108, 606. This can be achieved by forming the seals 110, 110′, 604, 604′ between the inner and outer layers (not shown) of the balloon 600 such that fluid is prevented from entering the tubular connectors 108, 606, and by providing separate inflation lumens to each of the distending members 104, 106, 602. (The function of the balloon 600 is substantially similar to the function of the balloon 102, discussed with reference to FIG. 4.) FIG. 7 illustrates another embodiment of a distending balloon 700 in an inflated state. The balloon 700 comprises a first distending member 104, a second distending member 702, and a cone-shaped tubular connector 704. The second distending member 702 has a diameter that is smaller than the diameter of the first distending member 104. Correspondingly, the distal end of the cone-shaped tubular connector 704 is smaller than the proximal end of the tubular connector 704. The cone-shaped tubular connector 704 interconnects the distending members 104, 702. As with the embodiments discussed above, in the embodiment of FIG. 7, the distending members 104, 702 and the cone-shaped tubular connector 704 may be made of a single, continuous one-piece balloon member that provides at least one interior inflatable chamber. An annular seal 708 is formed between the tubular connector 704 and the second distending member 702, and the annular seal 110 is formed between the tubular connector 704 and the first distending member 104. As with embodiments discussed above, the annular seals 110, 708 are formed circumferentially between inner and outer layers (not shown) of the balloon 700 using radio frequency (RF) welding, ultrasound welding, thermal bonding, adhesive, or other suitable sealing techniques.

The cone-shaped tubular connector 704 comprises inner and outer layers of the balloon 700, a plurality of support ribs 120 (such as illustrated above in FIGS. 1 and 3B), and a plurality of supportive depressions 706. In the embodiment illustrated in FIG. 7, it is contemplated that the support ribs 120 are distributed uniformly around the circumference of the cone-shaped tubular connector 704, and are oriented parallel with the inner and outer layers of the cone-shaped tubular connector 704. The support ribs 120 are held in position by the supportive depressions 706 and the annular seals 708, 110. The support ribs 120 and the supportive depressions 706 maintain the cone-shaped configuration of the tubular connector 704 when the balloon 700 supports a body cavity.

The supportive depressions 706 are localized regions of the tubular connector 704 in which the inner and outer layers (not shown) of the balloon 700 are adhered or bonded together. In another embodiment, the supportive depressions 706 may be holes which allow medical instruments, such as an endoscope, to pass unimpeded through the inner and outer layers of the tubular connector 704. Furthermore, the supportive depressions 706 may advantageously be implemented such that openings and/or window are incorporated into the cone-shaped tubular connector 704 as discussed with reference to FIGS. 1 through 3B. The supportive depressions 706 are formed by using radio frequency (RF) welding, ultrasound welding, thermal bonding, adhesive, or other suitable bonding techniques.

Additionally, in a preferred embodiment the supportive depressions 706 are uniformly distributed around the cone-shaped tubular connector 704, and the diameters of the supportive depressions 706 are directly proportional to the exterior diameter of the cone-shaped tubular connector 704. Specifically, the diameters of the supportive depressions 706 decrease in passing from a proximal end to a distal end of the cone-shaped tubular connector 704, thereby providing for an equal number of supportive depressions 706 on each end of the cone-shaped tubular connector 704. In another embodiment, however, the supportive depressions 706 may all have one size, thereby providing for fewer supportive depressions 706 on the distal end than on the proximal end of the cone-shaped tubular connector 704. Those of ordinary skill in the art will realize that the shapes, sizes and quantity of the supportive depressions 706 incorporated into the cone-shaped tubular connector 704 may advantageously be changed without detracting from the invention.

As further illustrated in FIG. 7, the first and second inflation tubes 116, 116′ are coupled to the balloon 700 as discussed above with reference to FIG. 1. It is contemplated that the first inflation tube 116 is used to inflate the first distending member 104 and the cone-shaped tubular connector 704, while the second inflation tube 116′ is used to inflate the second distending member 702. As discussed with reference to FIGS. 1 and 6, the first and second inflation tubes 116, 116′ of FIG. 7, as well as other inflation tubes that may optionally be included, can each accommodate a plurality of inflation lumens (not shown). For example, in other embodiments additional lumens and/or inflation tubes may be utilized such that the distending members 104, 702 and the cone-shaped tubular connector 704 can be inflated independently of each other. A person of ordinary skill in the art will recognize that the number of inflation tubes and the numbers of lumens therein may advantageously be changed without detracting from the invention.

Another embodiment of the balloon 700 may advantageously be constructed such that the distending members 104, 702 can be inflated without inflating the cone-shaped tubular connector 704. Specifically, as illustrated in FIG. 7, the annular seal 110 can be formed such that fluid is prevented from flowing into the cone-shaped tubular connector 704. (The function of the balloon 700 is substantially similar to the function of the balloon 102, discussed with reference to FIG. 4.)

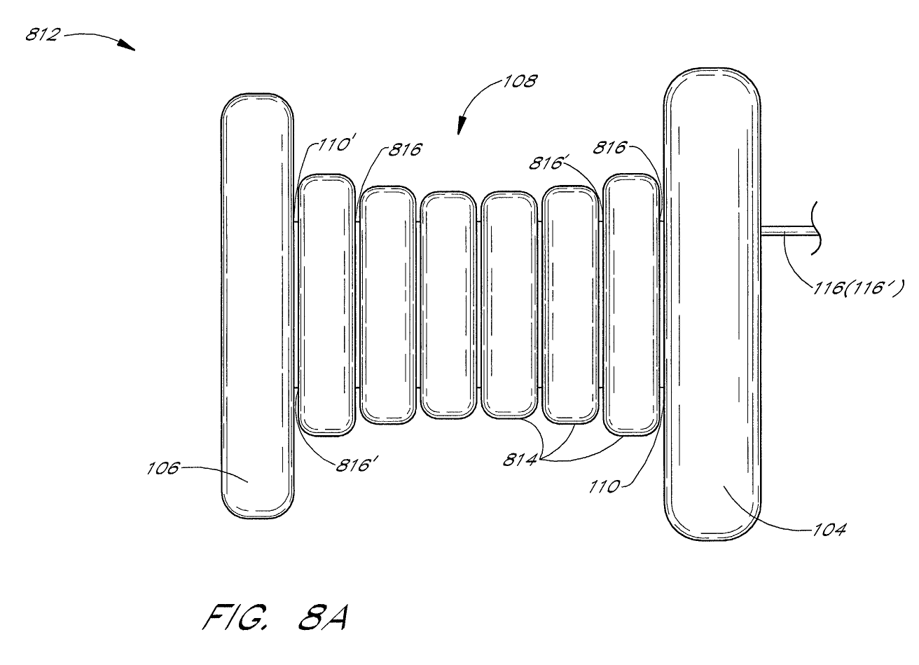

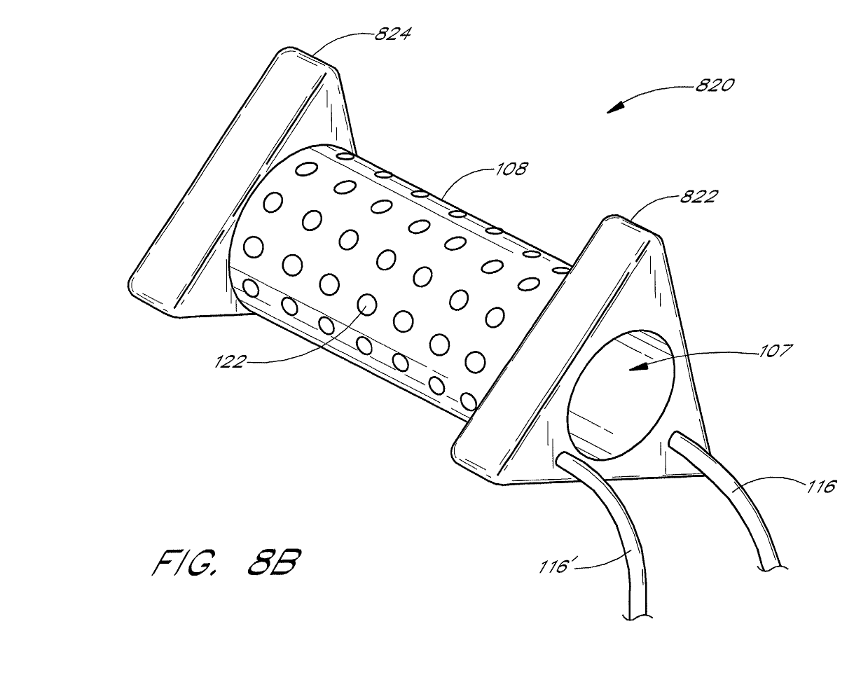

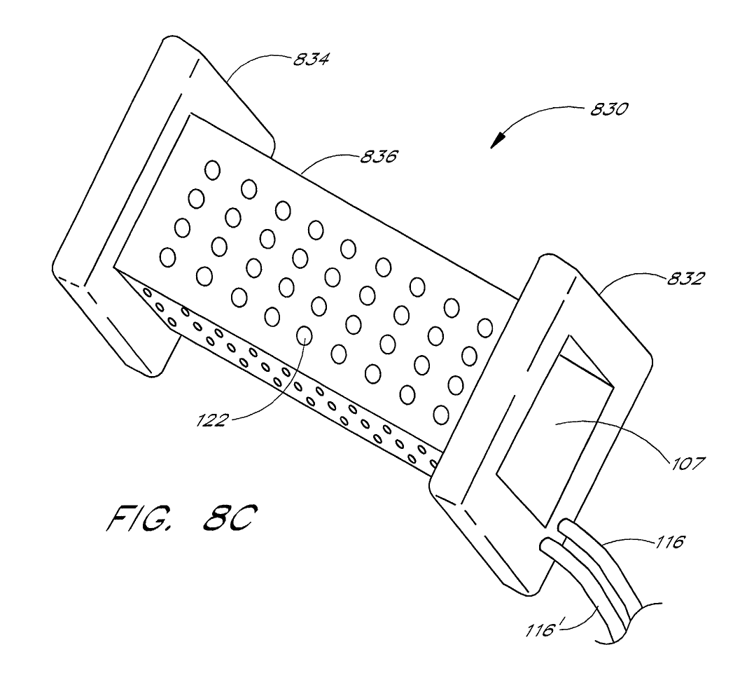

FIG. 8 illustrates another embodiment of a distending balloon 800 in an inflated state. The distending balloon 800 is substantially similar to the distending balloon 700 of FIG. 7, with the exception of an auxiliary distending member 802 and a narrow tubular connector 804. The cone-shaped tubular connector 704 interconnects the first distending member 104 and the auxiliary distending member 802. Similarly, the narrow tubular connector 804 interconnects the auxiliary and second distending members 802, 702. As with the embodiment of FIG. 7, in the embodiment of FIG. 8 the distending members 104, 802, 702 and the tubular connectors 704, 804 may be made of a single, continuous one-piece balloon member providing at least one interior inflatable chamber. An annular seal 808 is formed between the narrow tubular connector 804 and the auxiliary distending member 802, and an annular seal 808′ is formed between the auxiliary distending member 802 and the cone-shaped tubular connector 704. The annular seal 708 is formed between the narrow tubular connector 804 and the second distending member 702. The annular seals 808, 808′ are formed circumferentially between inner and outer layers (not shown) of the balloon 800 using radio frequency (RF) welding, ultrasound welding, thermal bonding, adhesive, or other suitable sealing techniques.

In the embodiment illustrated in FIG. 8, it is contemplated that the construction of the narrow tubular connector 804 is substantially similar to the construction of the tubular connector 108 (illustrated in FIGS. 1 though 3B). More specifically, the narrow tubular connector 804 comprises inner and outer layers of the balloon 800, wherebetween a plurality of support ribs 120 (such as illustrated in FIGS. 1 and 3B) are uniformly distributed around the circumference of the narrow tubular connector 804, and oriented parallel to the tubular connector 804. The support ribs 120 are held in position by a plurality of supportive depressions 806 and the annular seals 708, 808. The support ribs 120 and the supportive depressions 806 maintain an essentially cylindrical configuration of the narrow tubular connector 804 when the balloon 800 supports a body cavity. In one embodiment, a diameter of the supportive depressions 806 is directly proportional to a diameter of the narrow tubular connector 804. In another embodiment, the diameter of the supportive depressions 806 may be determined such that a specific number of depressions can be uniformly distributed around the circumference of the narrow tubular connector 804. Those of ordinary skill in the art will realize that the size and quantity of supportive depressions 806 utilized on the narrow tubular connector 804 may be changed without detracting from the invention.

As illustrated in FIG. 8, the first and second inflation tubes 116, 116′ are coupled to the balloon 800 as discussed above with reference to FIG. 1. It is contemplated that the first inflation tube 116 is used to inflate the first distending member 104 and the coneshaped tubular connector 704 while the second inflation tube 116′ is used to inflate the auxiliary distending member 802, the narrow tubular connector 804, and the second distending member 702. In this embodiment, the seals 110, 808, and 708 each has an opening to allow fluid communication between adjacent chambers. It will be recognized, however, that the first and second inflation tubes 116, 116′ can each accommodate a plurality of inflation lumens (not shown). For example, additional lumens may be utilized such that the distending members 104, 802, 702 and the tubular connectors 704, 804 can be inflated independently of each other when each of the seals between adjacent chambers is completely closed. Alternatively, this may be achieved by utilizing additional inflation tubes. Those of ordinary skill in the art will recognize that the number of inflation tubes, as well as the numbers of lumens therein, may advantageously be changed without detracting from the invention.

In another embodiment, the balloon 800 can be constructed such that the distending members 104, 802, 702 can be inflated without inflating the tubular connectors 704, 804. With this embodiment, the seals 110, 808, 808′, 708 are formed between the inner and outer layers (not shown) of the balloon 800 such that fluid is prevented from entering the tubular connectors 704, 804. (The function of the distending balloon 800 is substantially similar to the function of the balloon 102, discussed with reference to FIG. 4.)

FIG. 8A illustrates another embodiment of a distending balloon 812 in an inflated state. The balloon 812 comprises first and second distending members 104, 106, and a tubular connector 108 comprising a plurality of intermediate distending members 814. The intermediate distending members 814 preferably have diameters that are smaller than the diameters of the first and second distending members 104, 106. As with the embodiments discussed above, in the embodiment of FIG. 8A, it is contemplated that the distending members 104, 106 and the intermediate distending members 814 are made of a single, continuous one-piece balloon member that provides at least one interior inflatable chamber. An annular seal 110′ may be formed between the tubular connector 108 and the second distending member 106, and an annular seal 110 may be formed between the tubular connector 108 and the first distending member 104. Similarly, each intermediate distending member 814 may have a proximal annular seal 816 and a distal annular seal 816′ to isolate a chamber therebetween. The annular seals 110, 110′, 816, 816′ are formed circumferentially between inner and outer layers (not shown) of the balloon 812 using radio frequency (RF) welding, ultrasound welding, thermal bonding, adhesive, or other suitable sealing techniques. In the illustrated embodiment, it is contemplated that the annular seals 110, 816, 816′ may each include a small duct or unsealed passage that allows for fluid communication between the first distending member 104 and the intermediate distending members 814, thereby allowing the first distending member 104 and the intermediate distending members 814 to be inflated with one inflation tube, and the second distending member 106 to be inflated with a second inflation tube.

As illustrated in FIG. 8A, the first distending member 104 has a width that is greater than the width of the second distending member 106, and the width of the second distending member 106 is greater than the widths of the intermediate distending members 814. Additionally, the intermediate distending members 814 have diameters that decrease in passing from the first distending member 104 to the center of the tubular connector 108 and then increase in passing from the center of the tubular connector 108 to the second distending member 106. A person of ordinary skill in the art will recognize that in other embodiments, the relative widths and diameters of the distending members 104, 106, 814 may advantageously be determined based on a particular procedure contemplated, and as such may be substantially changed without detracting from the invention.

As further illustrated in FIG. 8A, the first and second inflation tubes 116, 116′ are coupled to the balloon 812 as discussed above with reference to FIG. 1. It is contemplated that the first inflation tube 116 is used to inflate the first distending member 104 and the intermediate distending members 814 while the second inflation tube 116′ is used to inflate the second distending member 106. It will be recognized, however, that the first and second inflation tubes 116, 116′ can each accommodate a plurality of inflation lumens (not shown). For example, additional lumens may be utilized such that the distending members 104, 106, 814 can be inflated independently of each other when each of the members are completely sealed off with respect to one another. This may alternatively be achieved by utilizing additional inflation tubes. Those of ordinary skill in the art will recognize that the number of inflation tubes, as well as the numbers of lumens therein, may advantageously be changed without detracting from the invention.

FIG. 8B illustrates another embodiment of a distending balloon 820 in an inflated state. The balloon 820 comprises first and second distending members 822, 824, a tubular connector 108, and a central lumen 107. The distending balloon 820 is substantially similar in construction to that of the distending balloon 102 of FIGS. 1 through 3B, except that the balloon 820 has distending members 822, 824 that are essentially triangular. As with the embodiments discussed above, in the embodiment of FIG. 8B, it is contemplated that the distending members 822, 824 and the tubular connector 108 are made of a single, continuous one-piece balloon member that provides at least one interior inflatable chamber. As further illustrated in FIG. 8B, the first and second inflation tubes 116, 116′ are coupled to the balloon 820 as discussed above with reference to FIG. 1. It is contemplated that the first inflation tube 116 is used to inflate the first distending member 822 and the tubular connector 108 while the second inflation tube 116′ is used to inflate the second distending member 824. The function of the balloon 820 is substantially similar to the function of the balloon 102.

FIG. 8C illustrates another embodiment of a distending balloon 830 in an inflated state. The balloon 830 comprises first and second distending members 832, 834, and a tubular connector 836. The distending balloon 830 is substantially similar in construction to that of the distending balloon 820 of FIG. 8B, except that the balloon 830 has distending members 832, 834 and a tubular connector 836 that are diamond-shaped. As with the embodiments discussed above, in the embodiment of FIG. 8C, it is contemplated that the distending members 832, 834 and the tubular connector 836 are made of a single, continuous one-piece balloon member that provides at least one interior inflatable chamber. Also illustrated in FIG. 8C, the first and second inflation tubes 116, 116′ are coupled to the balloon 830 as discussed above with reference to FIG. 1. It is contemplated that the first inflation tube 116 is used to inflate the first distending member 832 and the tubular connector 836 while the second inflation tube 116′ is used to inflate the second distending member 834. The function of the balloon 830 is substantially similar to the function of the balloon 102.

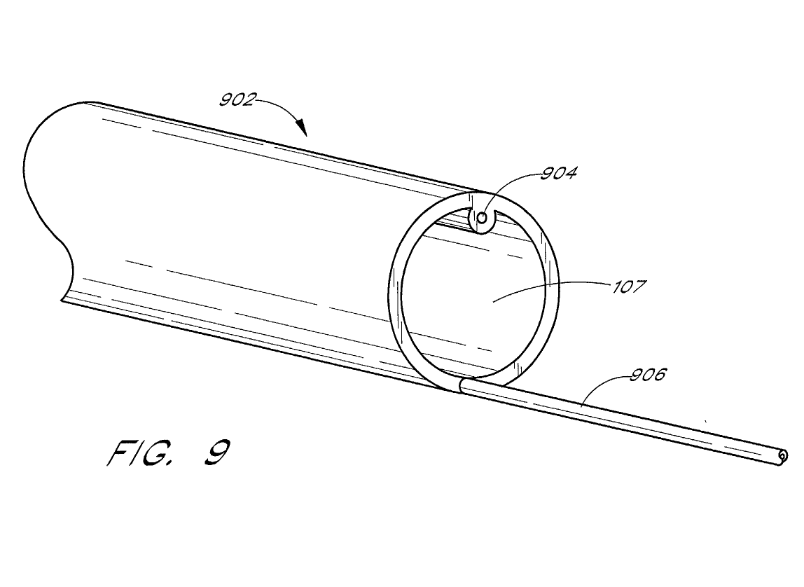

FIG. 9 illustrates another embodiment of a distending balloon 902 in an inflated state. The balloon 902 comprises a central lumen 107 and an auxiliary lumen 904. The balloon 902 is attached to an inflation tube 906, which is in fluid communication with the balloon 902. In another embodiment, a plurality of inflation tubes 906 may be attached to the balloon 902. In still another embodiment, the inflation tube 906 may accommodate a plurality of lumens.

The distending balloon 902 illustrated in FIG. 9 is preferably made of flexible, semi-compliant material. In one embodiment, the semi-compliant material allows the balloon 902 to expand about 1-20% upon being inflated to an optimal inflated state. In another embodiment, the semi-compliant material allows the balloon 902 to expand about 1-15% upon inflation to an optimal inflated state. In still another embodiment, the semi-compliant material allows the balloon 902 to expand about 1-10% upon being inflated to an optimal inflated state. In yet another embodiment, the semi-compliant material allows the balloon 902 to expand about 1-5% upon inflation to an optimal inflated state. Additionally, the flexibility of the material facilitates bending and inserting the balloon 902 in various regions of a patient's body. In one embodiment, the balloon 902 is made of polyurethane. In another embodiment, the balloon 902 may be made of polypropylene. In still another embodiment, the balloon 902 may be made of silicone. Other materials include other non-compliant or semi-compliant materials, or blends thereof, including but not limited to EVA (Ethylene-Vinyl-Acetate), PVC, PET, and NYLON. Those of ordinary skill in the art will recognize that the balloon 902 may advantageously be made of other non-compliant or semi-compliant, biocompatible materials without detracting from the invention.

Alternatively, the balloon 902, or portions thereof, may advantageously be made of a transparent or translucent material to facilitate visual inspections of body cavities. In one embodiment, specific portions of the balloon 902 are made of transparent material. In another embodiment, the entirety of the balloon 902 is made of transparent material. In still another embodiment, specific portions of the balloon 902 are made of translucent material. In yet another embodiment, the entirety of the balloon 902 is made of translucent material. A person of ordinary skill in the art will realize that the opacity of the balloon 902, or individual portions thereof, may advantageously be changed without detracting from the invention.

In a preferred embodiment, the diameter of the central lumen 107 is sufficiently large to allow a physician to insert one or more medical instruments through the central lumen 107. The auxiliary lumen 904 is sized to receive medical devices, such as a guide wire, an endoscope, or other instrument (not shown). In one embodiment, the tube forming the auxiliary lumen 904 may be less compliant (i.e., more rigid) than the material of the balloon 902. In this embodiment, the tube forming the auxiliary lumen 904 may be molded, bonded, or otherwise attached to the surface of the central lumen 107.

In operation, a physician places the distending balloon 902 in a deflated or semi-inflated state and then inserts the balloon 902 into a cavity of a patient's body that is to be enlarged, or distended, and supported. Such insertion may be assisted by inserting a guide wire, or other similar delivery system, into the cavity of the patient and advancing the auxiliary lumen 904 over the guide wire to guide the insertion and placement of the balloon 902. The auxiliary lumen 904 may also be used for diagnostic purposes. In one embodiment, the balloon 902 in the deflated state is rolled into a long, thin configuration to facilitate insertion into a body cavity. In another embodiment, the balloon 902 may be used in conjunction with a balloon applicator to facilitate insertion into a body cavity. Balloon applicators will be discussed in greater detail below.

Once the distending balloon 902 is inserted and placed in a desired position within the body cavity, the physician inflates the balloon 902 via the inflation tube 906 with saline solution, water, air, or other suitable fluid. The proximal end of the inflation tube 906 extends from the balloon 902 for connection to a source of fluid, such as a syringe. The balloon 902 is sized such that, as the balloon 902 inflates to an optimal inflated state, the outer surface of the balloon 902 exerts pressure on the interior surface of the body cavity, thereby supporting the body cavity in a distended state.

When the balloon 902 reaches the optimal inflated state, as shown in FIG. 9, the physician ceases inflation of the balloon 902. In one embodiment, the physician uses a pressure-measuring device (not shown) coupled to the inflation tube 906 to determine when the balloon 902 reaches the optimal inflated state. In another embodiment, an over-inflation balloon may advantageously be used as discussed with reference to FIG. 1.

When the balloon 902 is in the inflated state, medical instruments, such as an endoscope, or biological material, such as blood, may pass from one end of the balloon 902 through the central lumen 107 to the other end of the balloon 902. Thus, the central lumen 107 advantageously allows material and objects to pass through the balloon 902 unimpeded while the balloon 902 enlarges, and supports the body cavity in the distended state. In one application, where the balloon 902 is used to expand a patient's vagina 404, instruments may be passed through the central lumen 107 to perform a medical procedure, such as tissue sampling or a Pap smear.

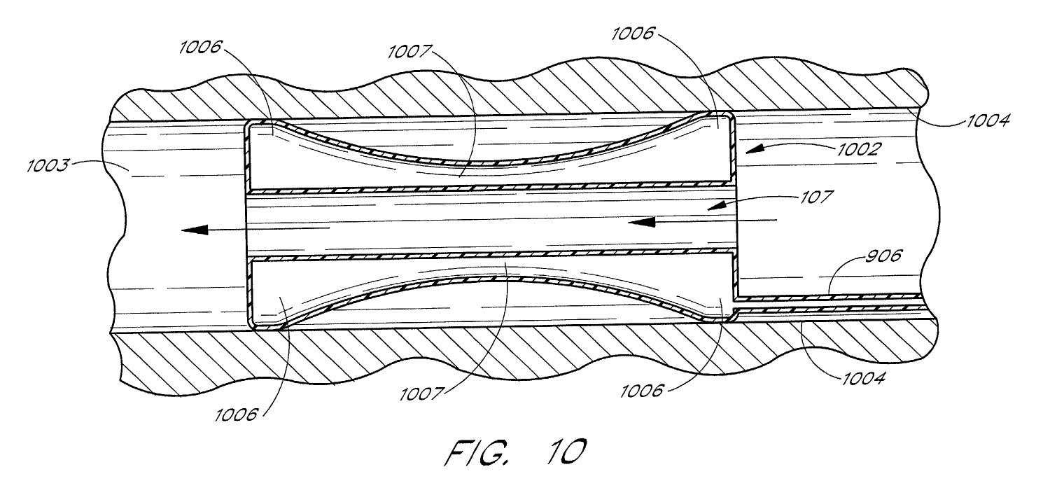

FIG. 10 is a cross-sectional side view of another embodiment of a distending balloon 1002 in an inflated state. As illustrated in FIG. 10, the balloon 1002 is supporting a body cavity 1003, having side walls 1004, in a distended state. The structure of the balloon 1002 is substantially similar to the structure of the balloon 902 shown in FIG. 9, with the exception that the balloon 1002 comprises enlarged annular end portions 1006, which are interconnected by an intermediate portion 1007. When the balloon 1002 is inflated to an optimal inflated state, the enlarged end portions 1006 extend radially outward beyond the intermediate portion 1007 such that most, or substantially all, of the force against the walls 1004 of the body cavity 1003 is provided by the enlarged end portions 1006. While the inflated balloon 1002 supports the body cavity 1003, the enlarged end portions 1006 hold the balloon 1002 in place, thereby minimizing the movement of the balloon 1002 relative to the body cavity 1003.

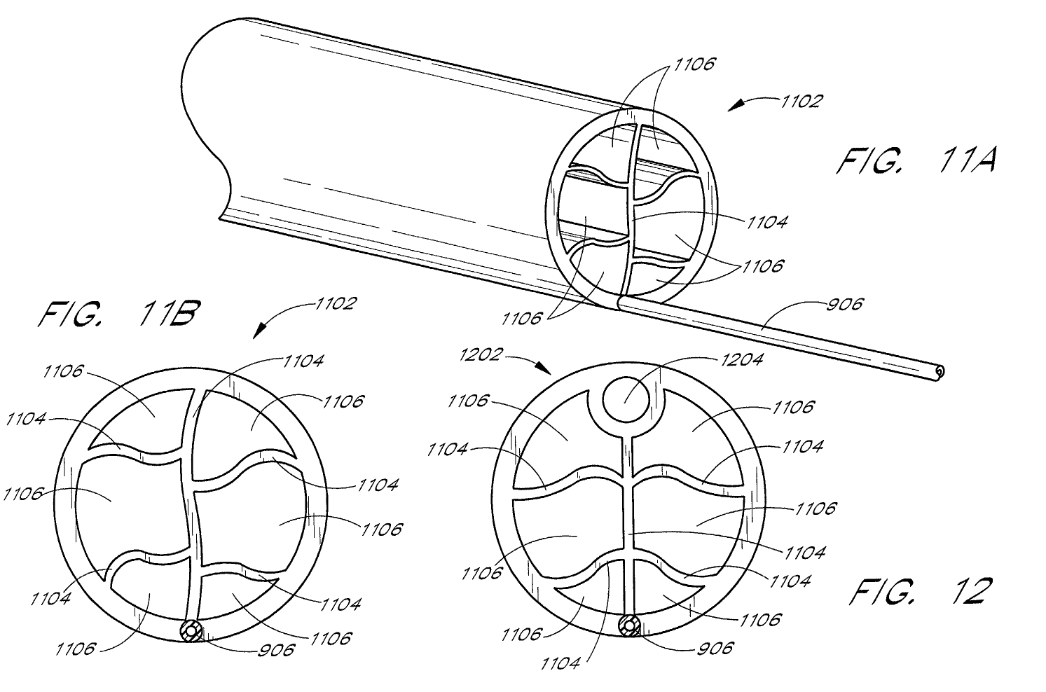

FIGS. 11A and 11B illustrate another embodiment of a distending balloon 1102 in an inflated state. The distending balloon 1102 has substantially the same structure as the balloon 902 shown in FIG. 9, with the exception that the balloon 1102 comprises a plurality of interconnected internal walls 1104 which form a plurality of lumens 1106. In one embodiment, the walls 1104 are made of the same material as the balloon 1102. In another embodiment, the walls 1104 are made of a less compliant and/or less flexible (i.e., more rigid) material than the balloon 1102. The walls 1104 may support the shape of the balloon 1102 as the balloon 1102 inflates. In still another embodiment, the walls 1104 are substantially non-compliant to prevent the balloon 1102 from expanding beyond an optimal inflation state, as shown in FIG. 11A.

The lumens 1106 allow biological material such as blood to flow through the distending balloon 1102. The lumens 1106 may be round or angular in shape. In one embodiment, the lumens 1106 are adapted to allow a physician to pass medical instruments through one or more of the lumens 1106 of the balloon 1102.

FIG. 12 is a cross-sectional view of another embodiment of a distending balloon 1202 in an inflated state. The distending balloon 1202 has substantially the same structure as the distending balloon 1102 illustrated in FIGS. 11A and 11B, except that the balloon 1202 comprises an additional, auxiliary lumen 1204 which is similar to the auxiliary lumen 904 illustrated in FIG. 9. As described above with reference to FIG. 9, the auxiliary lumen 1204 is adapted to receive a guide wire, an endoscope, or other narrow instrument (not shown). In one embodiment, the tube forming the auxiliary lumen 1204 may be less compliant and/or less flexible (i.e., more rigid) than the material of the balloon 1202. In this embodiment, the tube forming the auxiliary lumen 1204 may be molded, bonded or otherwise attached to the distending balloon 1202.

FIG. 13 is a cross-sectional view of another embodiment of a distending balloon 1302 in an inflated state. The structure of the balloon 1302 is substantially similar to the structure of the balloon 902 illustrated in FIG. 11B, with the exception that the balloon 1302 comprises a plurality of lumens 1304 having substantially round cross sections. The function of the balloon 1302 is substantially similar to the function of the balloon 902 in FIG. 11B, as described above.

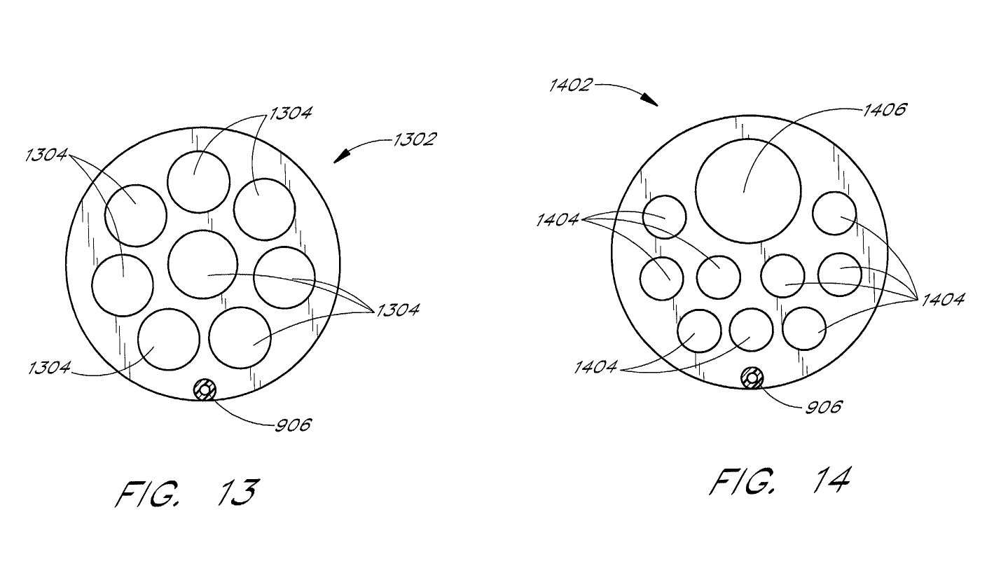

FIG. 14 is a cross-sectional view of another embodiment of a distending balloon 1402 in an inflated state. The distending balloon 1402 of FIG. 14 is substantially similar in structure to the balloon 1302 in FIG. 13, with the exception that the balloon 1402 comprises a plurality of smaller lumens 1404 and a primary lumen 1406. The primary lumen 1406 is similar to the auxiliary lumen 904 illustrated in FIG. 9. As with the auxiliary lumen 904, the primary lumen 1406 is adapted to receive a guide wire, an endoscope, or other narrow instrument (not shown). In one embodiment, the tube forming the primary lumen 1406 may be less compliant and/or less flexible (i.e., more rigid) than the material of the balloon 1402. In this embodiment, the tube forming the primary lumen 1406 may be molded, bonded, or otherwise incorporated into the balloon 1402. The function of the balloon 1402 in FIG. 14 is substantially similar to the function of the balloon 902 in FIG. 11B, as described above.

In the embodiments discussed with reference to FIGS. 9 through 14, the inflation tube 906 may extend the entire length of the distending balloon. Like the auxiliary lumen 904, the inflation tube 906 may be formed of a material that is rigid compared to the flexible balloon material. The flexible balloon material may be wrapped around the rigid material, and the rigid material may be used as a supportive structure for inserting the balloon into a body cavity. Preferably the rigid material has a degree of flexibility so as to allow the balloon to follow any curvature in the body cavity, particularly if the body cavity is a lumen or channel.

FIG. 15 is a side view of one embodiment of a balloon applicator 1500 that is used for inserting the distending balloon 102 such as illustrated in FIGS. 1 through 3B into a body cavity. It will be appreciated that the balloon applicator may also be used to insert the other balloons described above. The balloon applicator 1500 preferably comprises a shaft section 1502, a curved retainer 1504, and a handle section 1506. As is shown in FIG. 15, the shaft section 1502 interconnects the curved retainer 1504 and the handle section 1506, such that the three sections are preferably integrally formed. The curved retainer 1504 facilitates mounting and maintaining the distending balloon 102 on the applicator 1500 in a deflated, folded state. The handle section 1506 facilitates holding the applicator 1500 during operation. In one embodiment, the balloon applicator 1500 is made of a metal, such as steel. In another embodiment, the balloon applicator 1500 may be made of a rigid material, such as hard plastic or metal, so as to prevent bending of the shaft section 1502 during operation.

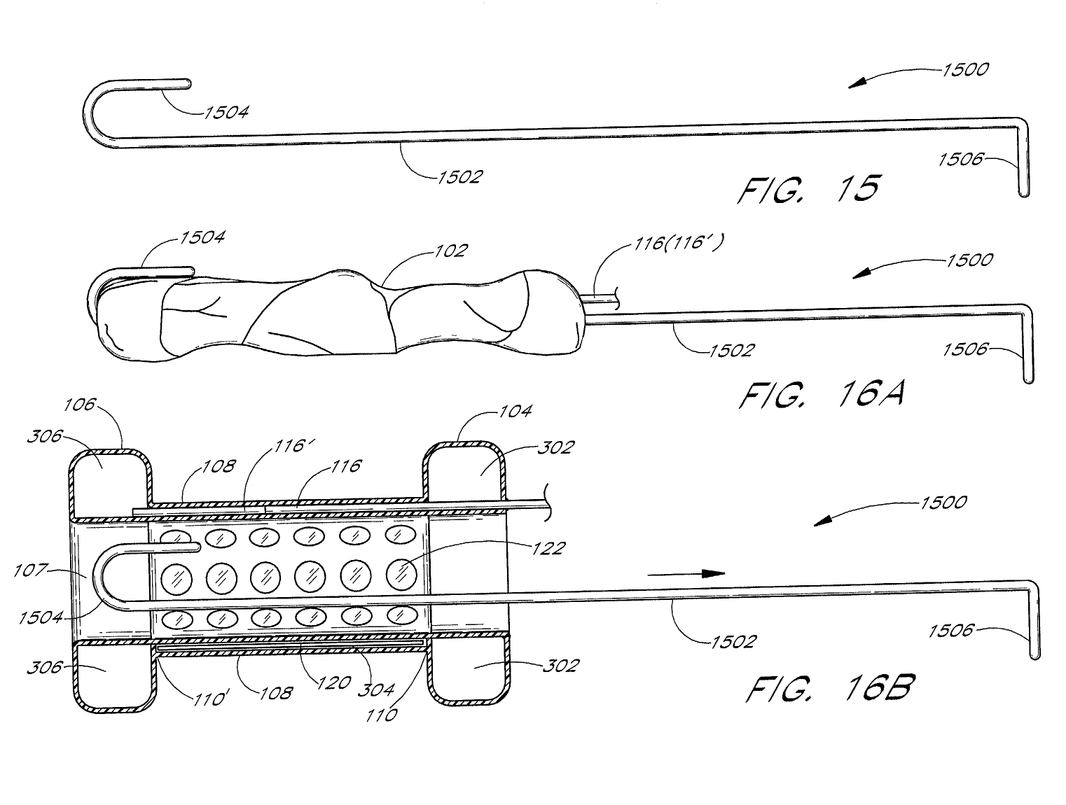

FIGS. 16A and 16B generally illustrate the use of the balloon applicator 1500 as used for inserting the distending balloon 102 into a body cavity. Referring to FIG. 16A, a physician preferably deflates the distending balloon 102 and then applies a lubricant to the balloon 102 to prevent the exterior surfaces of the balloon 102 from sticking together when inserted into the body cavity. Next, the physician inserts the applicator 1500 into the central lumen 107 of the balloon 102 and then tightly folds the balloon 102 around the shaft section 1502 of the balloon applicator 1500 placing the balloon 102 into a narrow, folded state. The physician then slides the balloon 102 distally on the shaft section 1502, thereby moving the distal portion of the balloon 102 within the curved retainer 1504. Although the curved retainer 1504 serves to hold the balloon 102 in the narrow, wrapped state, the physician may optionally tack-weld the balloon 102 in the narrow, wrapped state to further prevent unraveling of the balloon 102 during the insertion process. The physician may also apply lubrication to the exterior of the balloon 102 in the narrow, folded state. The physician then inserts the balloon 102 and the balloon applicator 1500 into the body cavity.

Once the distending balloon 102 and the balloon applicator 1500 have been inserted into a desired position within a body cavity, the physician inflates the balloon 102 with saline solution or other suitable fluid, as discussed with reference to FIG. 4. When the balloon 102 begins to expand, the distal portion of the balloon slides out of the curved retainer 1504 and the balloon 102 smoothly unfolds. As the balloon 102 expands, it supports the body cavity in a distended state. Referring to FIG. 16B, once the balloon 102 has been inflated to an optimal inflated state, the physician moves the applicator 1500 proximally, thereby withdrawing the retaining hook 1504 from the patient's body cavity through the central lumen 107 of the balloon 102. With the balloon applicator 1500 removed from the balloon 102, the physician then performs medical procedures as discussed with reference to FIG. 4.

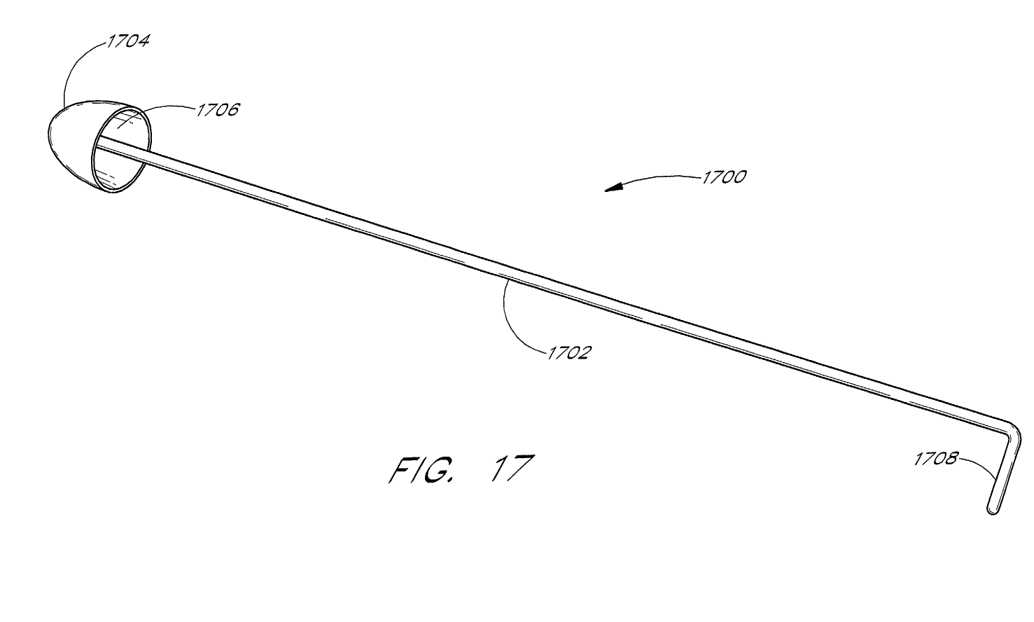

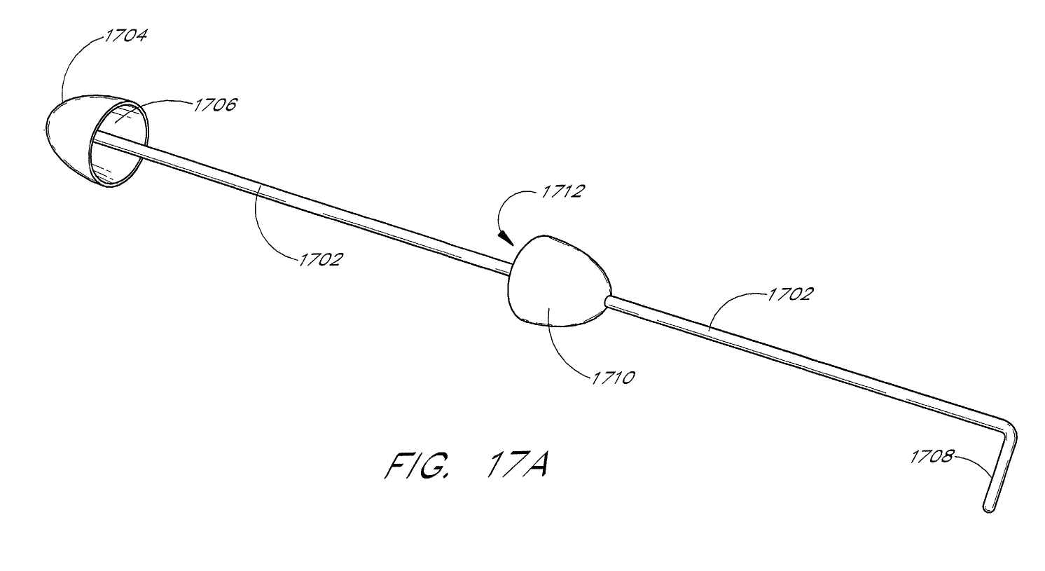

FIG. 17 is a perspective view of another embodiment of a balloon applicator 1700 that can be used for inserting the distending balloon 102 into a body cavity. The balloon applicator 1700 preferably comprises a shaft section 1702, a retaining bell 1704, and a handle section 1708. The retaining bell 1704 further comprises a retaining cavity 1706 which receives a distal end of the shaft section 1702. The retaining bell 1704 facilitates mounting and maintaining the distending balloon 102 on the balloon applicator 1700 in a narrow, wrapped configuration. The handle section 1708 facilitates holding the applicator 1700 during operation of the balloon applicator 1700. In one embodiment, the balloon applicator 1700 is made of a metal, such as steel. In another embodiment, the balloon applicator 1700 may be made of a rigid material, such as hard plastic, so as to prevent bending of the shaft section 1702 during operation. Furthermore, the balloon applicator 1700 illustrated in FIG. 17 is of a one-piece design. However, it will be realized by those skilled in the art that the retaining bell 1704, the shaft section 1702, and the handle section 1708 may be individual components which are separately manufactured and then assembled to create the balloon applicator 1700.

In another embodiment, the retaining bell 1704 can be made of a flexible material such that it stretches and then inverts when the balloon 102 is inflated to an optimal inflated state. Once the flexible retaining bell 1704 is inverted, and the balloon 102 is inflated to the optimal inflated state, the balloon applicator 1700 can be withdrawn from the body cavity through the central lumen 107.

FIG. 17A illustrates a slightly modified form of the balloon applicator 1700, wherein a secondary retaining bell 1710 is mounted on the shaft section 1702. The secondary retaining bell 1710 further comprises a retaining cavity 1712. The secondary retaining bell 1710 facilitates maintaining the proximal portion of the balloon 102 on the applicator 1700 in the narrow, folded configuration while the balloon 102 is being inserted into a body cavity. In one embodiment, the secondary retaining bell 1710 is fixed to the shaft section 1702. With this embodiment, the secondary retaining bell 1710 is spaced a distance apart from the retaining bell 1704 such that the distal and proximal portions of the balloon 102, in the narrow, folded configuration, can be tucked within the retaining cavities 1706, 1712, respectively. In another embodiment, the secondary retaining bell 1710 is slidably attached to the shaft section 1702. In this embodiment, the secondary retaining bell 1710 can be moved distally along the shaft section 1702, allowing the proximal portion of the balloon 102 to be tucked into the retaining cavity 1712.

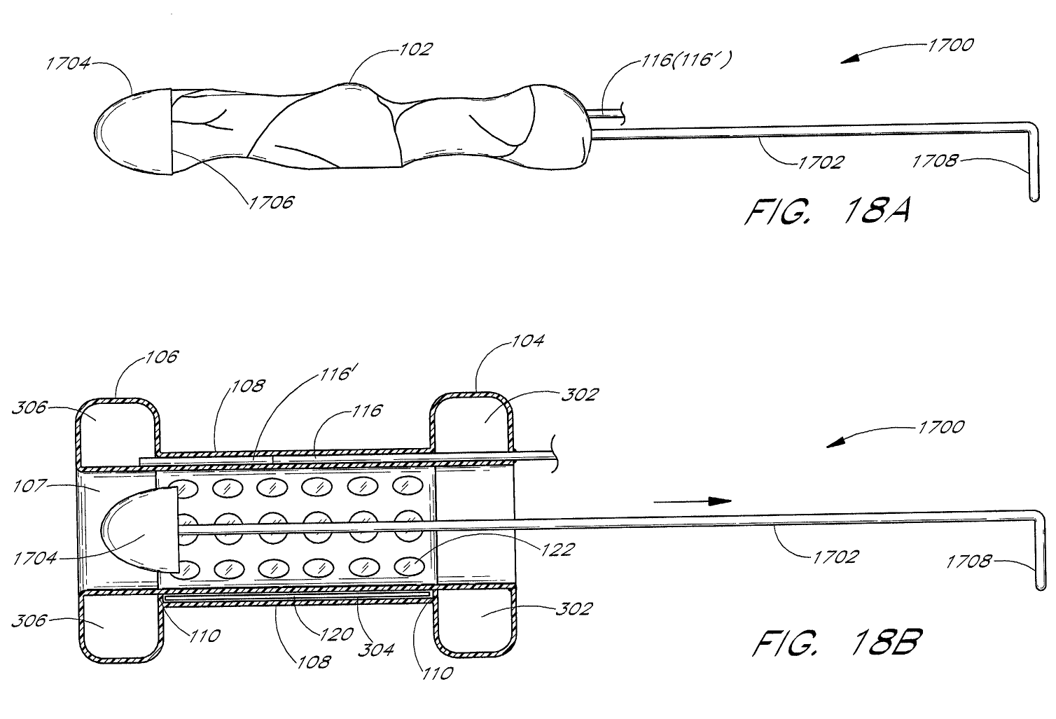

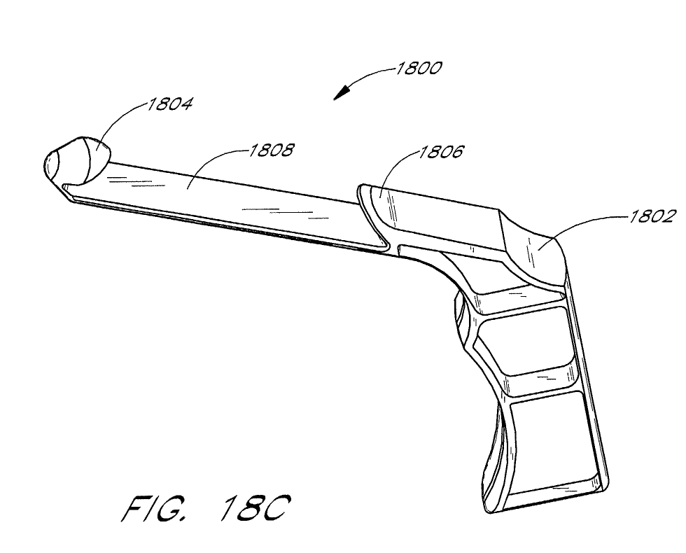

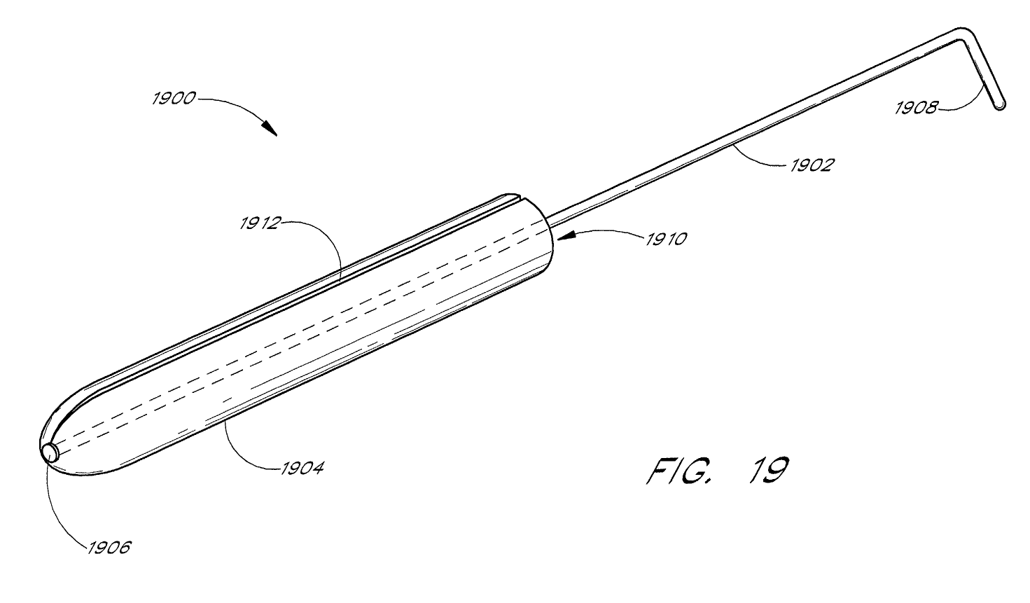

FIGS. 18A and 18B generally illustrate the use of the balloon applicator 1700, illustrated in FIG. 17, as used for inserting the distending balloon 102 into a body cavity. The function of the balloon applicator 1700 of FIG. 17 is substantially similar to the function of the balloon applicator 1500 of FIG. 15. Referring to FIG. 18A, a physician first deflates and lubricates the distending balloon 102, as discussed above. The physician then inserts the applicator 1800 into the central lumen 107 of the balloon 102 and then tightly folds the balloon 102 around the shaft section 1702, placing the balloon 102 into a narrow, folded configuration. Next, the physician slides the balloon 102 distally along the shaft section 1702, which moves the distal portion of the balloon 102 into the retaining cavity 1706. The physician may optionally tack-weld the balloon 102 in the narrow, wrapped configuration as a further precaution against unraveling of the balloon 102 during the insertion process. The physician may then apply lubrication to the exterior of the balloon 102 in the narrow, folded configuration. The physician can then use a finger to hold the proximal portion of the folded balloon 102 close to the shaft section 1702 of the applicator 1700 during insertion of the balloon 102 into the body cavity. Alternatively, the physician can use the balloon applicator 1700 illustrated in FIG. 17A, thereby avoiding the need for holding the balloon 102 with a finger.

The procedure used for withdrawing the balloon applicator 1700 from the body cavity is substantially similar to the procedure used to withdraw the balloon applicator 1500 of FIG. 15. Once the distending balloon 102 and the balloon applicator 1700 are positioned as desired within the body cavity, the physician inflates the balloon 102 with saline solution or other suitable fluid, as discussed with reference to FIG. 4. When the balloon 102 begins to expand, the distal portion of the balloon slides smoothly out of the retaining cavity 1706. As the balloon 102 expands, it supports the body cavity in a distended state. Referring to FIG. 18B, once the balloon 102 has been inflated to an optimal inflated state, the physician moves the applicator 1700 proximally, thereby withdrawing the retaining bell 1704 from the patient's body cavity through the central lumen 107 of the balloon 102. With the balloon applicator 1700 removed from the balloon 102, the physician then performs medical procedures as discussed in reference with FIG. 4.

FIG. 18C is a perspective view of another embodiment of a balloon applicator 1800 that is used for inserting the distending balloon 102 into a body cavity. The balloon applicator 1800 preferably comprises a handle section 1802, a distal retainer 1804, a proximal retainer 1806, and a balloon rest 1808. The distal and proximal retainers 1804, 1806 facilitate maintaining the balloon 102 is a narrow, folded configuration while the balloon 102 is being inserted into the body cavity. The balloon rest 1808 is a flat surface that provides lengthwise support for the folded balloon 102.

The function of the balloon applicator 1800 is substantially similar to the function of the balloon applicator 1500 illustrated in FIG. 15, with the exception that the applicator 1800 is not inserted into the central lumen 107 of the balloon 102. Rather, with the applicator 1800, a physician folds the balloon 102 lengthwise onto itself several times, thereby placing the balloon 102 into the narrow, folded configuration separately from the applicator 1800. Following this, the physician places the folded balloon 102 onto the balloon rest 1808, and then tucks the distal and proximal portions of the balloon 102 within the distal and proximal retainers 1804, 1806, respectively. The physician may optionally tack-weld the balloon 102 in the narrow, folded configuration as a further precaution against unfolding of the balloon 102 during the insertion process.

Once the balloon 102 and the balloon applicator 1800 are positioned within the body cavity, the physician inflates the balloon 102 with saline solution, or other suitable fluid, as discussed with reference to FIG. 4. When the balloon 102 begins to expand, the distal and proximal portions of the balloon 102 slide smoothly out of the distal and proximal retainers 1804, 1806. As the balloon 102 continues to expand, the physician withdraws the balloon applicator 1800 from the patient's body while the balloon 102 supports the body cavity in a distended state.

FIG. 19 is a perspective view of another embodiment of a balloon applicator 1900 that can be used for inserting the distending balloon 102 into a body cavity. The balloon applicator 1900 preferably comprises a shaft section 1902, a retaining sleeve 1904, a distal end 1906, and a handle section 1908. The retaining sleeve 1904 is preferably made of a semi-compliant material, such as polyurethane, polypropylene, or other suitable material. The retaining sleeve 1904 further comprises a retaining cavity 1910 and a tear-line 1912. The retaining cavity 1910 receives a distal portion of the shaft section 1902 and is fixedly attached to the distal end 1906. The handle section 1908 facilitates holding the applicator 1900 during use. In one embodiment, the shaft section 1902, the distal end 1906, and the handle section 1908 are made of a metal, such as steel. In another embodiment, the shaft and handle sections 1902, 1908 may be made of a substantially rigid material, such as hard plastic, so as to prevent bending during operation of the applicator 1900.

The retaining cavity 1910 maintains the distending balloon 102 in a deflated, wrapped state during use of the applicator 1900. The tear-line 1912 comprises a longitudinally oriented strip of the retaining sleeve 1904 wherein the thickness of the material comprising the retaining sleeve 1904 is substantially reduced. The tear-line 1912 allows the retaining sleeve 1904 to tear open when the distending balloon 102 is inflated. Those of ordinary skill in the art will realize that tearing open the retaining sleeve 1904 renders the retaining sleeve 1904 unusable. In one embodiment, the retaining sleeve 1904 is removable from the distal end 1906 of the shaft section 1902, thereby facilitating the replacement of torn retaining sleeves 1904. In another embodiment, the retaining sleeve 1904 is permanently fixed to the distal end 1906. In this embodiment, the balloon applicator 1900 is discarded after each use.

In another embodiment, the retaining sleeve 1904 may have a length that is substantially shorter than illustrated in FIG. 19. With this embodiment, the retaining sleeve 1904 does not tear open when the balloon 102 is inflated; rather, the retaining sleeve 1904 stretches into an umbrella-like configuration and then inverts, thereby avoiding the need for the tear-line 1912. The inverted retaining sleeve 1904 can then be withdrawn through the central lumen 107 of the balloon 102.

A person of ordinary skill in the art will realize that, in the embodiment of FIG. 19, the distending balloon 102 is preferably wrapped onto the shaft section 1902 and inserted into the retaining cavity 1910 by a practitioner of the invention. In this embodiment, the balloon applicator 1900 can be used in conjunction with a plurality of distending balloons 102. In another embodiment, a manufacturer of the balloon applicator 1900 may insert the distending balloon 102 into the retaining cavity 1910. With this embodiment, the practitioner merely selects a balloon applicator 1900 that has a distending balloon 102 that is appropriately sized for the particular medical procedure contemplated.

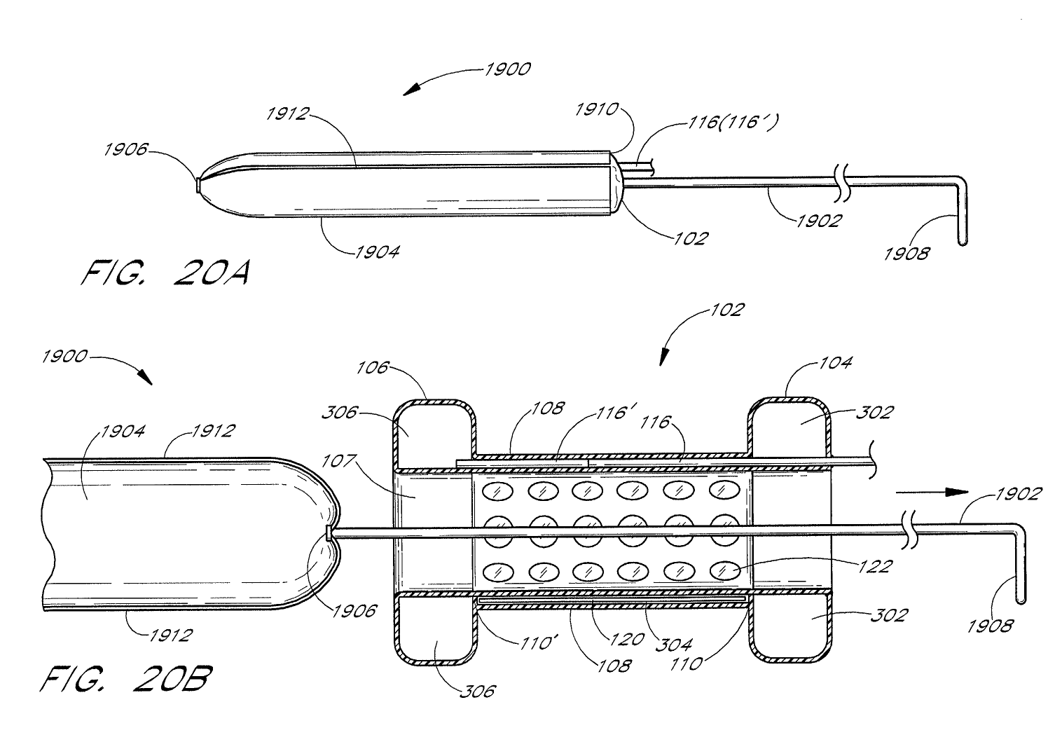

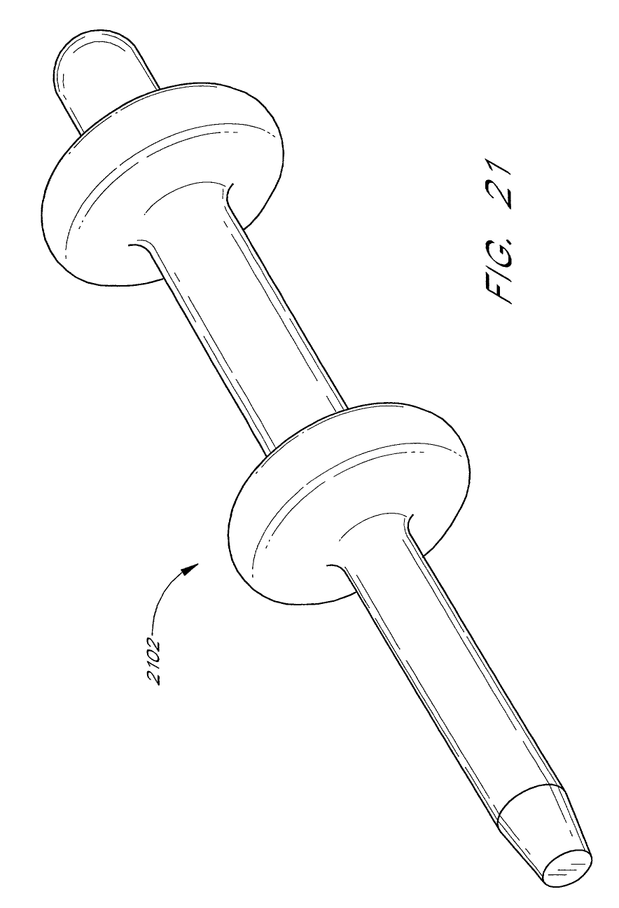

FIGS. 20A and 20B generally illustrate the use of the balloon applicator 1900 as used for inserting the distending balloon 102 into a body cavity. Referring to FIG. 20A, a physician prepares the distending balloon 102 as discussed above with reference to FIGS. 16A and 18A. Next, the physician inserts the applicator 1900 into the central lumen of the balloon 102 and then tightly folds the balloon 102 around the shaft section 1902. The physician may then apply lubrication to the exterior of the folded balloon 102 to facilitate sliding the balloon 102 into the retaining sleeve 1904. The physician then slides the folded balloon 102 distally along the shaft section 1902 and moves the entire length of the balloon 102 into the retaining cavity 1910.

A person of ordinary skill in the art will recognize that the steps required to prepare the balloon 102 and the balloon applicator 1900 may advantageously be avoided if the physician uses a balloon applicator 1900 having a manufacturer-inserted distending balloon 102. In this case, the physician need only select a balloon applicator 1900 that has a distending balloon 102 of the desired size.

Once the distending balloon 102 and the balloon applicator 1900 are positioned as desired within a body cavity, the physician inflates the balloon 102 with saline solution or other suitable fluid, as discussed with reference to FIG. 4. As the balloon 102 expands, it exerts pressure on the retaining sleeve 1904 and the body cavity. As the balloon 102 is further inflated, the retaining sleeve 1904 tears open along the tear-line 1912, allowing the balloon 102 to continue expanding the body cavity. Referring to FIG. 20B, once the balloon 102 has inflated to an optimal inflated state, the physician moves the applicator 1900 proximally, thereby withdrawing the shaft section 1902, the distal end 1906, and the torn retaining sleeve 1904 from the patient's body cavity through the central lumen 107 of the balloon 102. With the balloon applicator 1900 removed from the balloon 102, the physician then performs medical procedures as discussed in reference to FIG. 4.

Referring to FIGS. 21 through 23B, a preferred method for manufacturing the distending balloon 102, wherein a “dip-molding” process is utilized, will be discussed. It is to be understood, however, that a variety of other methods, such as, by way of example, “blow-molding,” may be utilized for manufacturing the balloon 102, as well as the other balloon embodiments disclosed herein, without detracting from the invention.

A mandrel 2102 may advantageously be used to manufacture a balloon member 2202. The mandrel 2102 is preferably composed of 304 (or higher) stainless steel that is electro-polished after machining. A person of ordinary skill in the art will realize that the mandrel 2102 may advantageously be made of other materials without detracting from the invention.

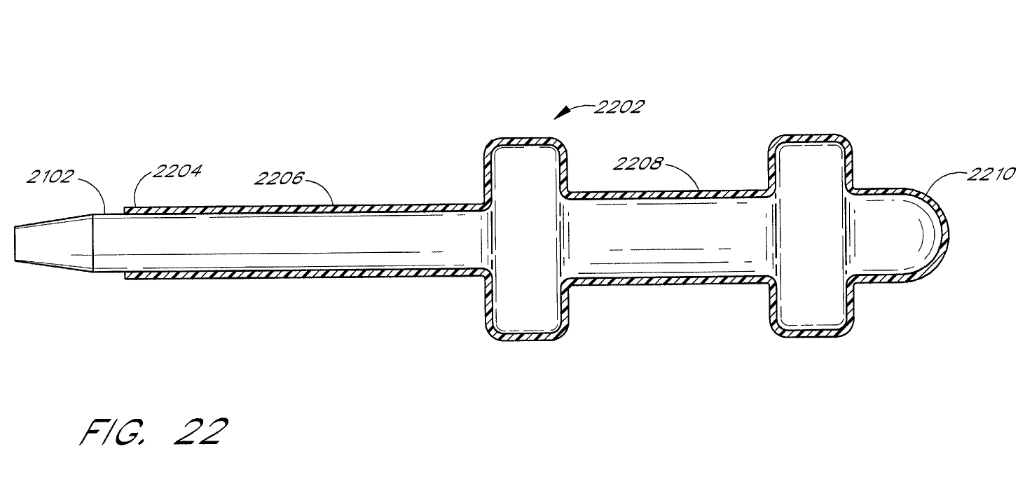

During the balloon manufacturing process, the mandrel 2102 is appropriately dipped in a liquid polyethylene, polyurethane or other solution of low compliance biocompatible material a sufficient number of times to produce a wall thickness of ranging between approximately 0.015 inches to 0.030 inches. The wall thicknesses illustrated in FIGS. 22 though 23B are exaggerated to facilitate visualization of the balloon's construction.

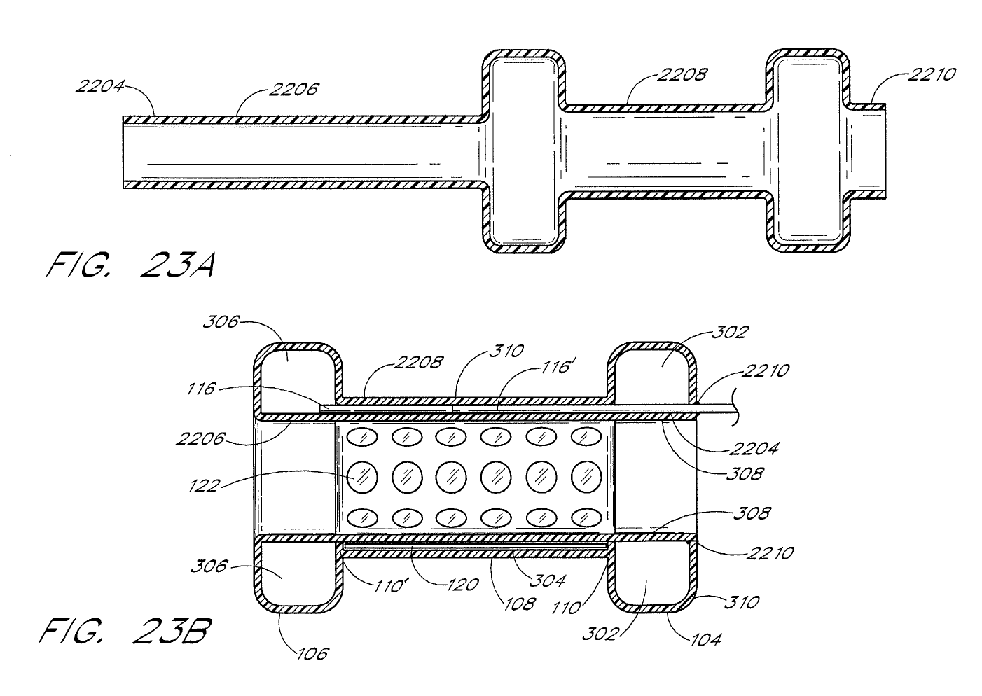

Following the dipping process, the balloon member 2202 is a single, continuous one-piece member having an open end 2204, a first elongated section 2206, a second elongated section 2208, and a rounded end portion 2210. The first elongated section 2206 is slightly smaller in diameter than the second elongated section 2208 as a result of a corresponding difference in the diameters of the respective mandrel sections. The balloon member 2208 is subsequently removed from the mandrel 2102. As illustrated in FIG. 23A, the rounded end portion 2210 is trimmed such that it is no longer enclosed but is open. As illustrated in FIG. 23B, the open end 2204 is then inverted inward, and the first elongated portion 2206 is pulled through the center of the balloon member 2202 such that the open end 2204 aligns with the trimmed rounded end 2210. In so doing, the first elongated section 2206 forms the inner layer 308 of the balloon 102 and the second elongated section 2208 forms the outer layer 310 of the balloon 102. Because the first elongated section 2206 is smaller in diameter than the second elongated section 2208, the first elongated section fits within the second section.

Once the first elongated section 2206 is pulled through the second elongated section 2208, the portions of the inner and outer layers 308, 310 forming the tubular connector 108 are adhered together in a plurality of locations to form the supportive depressions 122. The inflation tubes 116, 116′ are then inserted between the inner and outer layers 308, 310, and the supportive depressions 122. The inflation tubes 116, 116′ are preferably formed of a semi-rigid, translucent material such as polyethylene. In a preferred embodiment, the inflation tube 116 is inserted to a distance such that the inflation lumen 112 (FIG. 1) opens into the central inflation chamber 304. Similarly, the inflation tube 116′ is inserted such that the inflation lumen 114 (FIG. 1) opens into the second inflation chamber 306. Next, the support ribs 120 are inserted between the inner and outer layers 308, 310, and the supportive depressions 122, as discussed with reference to FIGS. 3A and 3B. Thereafter, the edges of the open end 2204 and the rounded end 2210 are circumferentially sealed to one another using known sealing methods, such as RF welding, thermal bonding or adhesives. Once sealed, the open end 2204 and the trimmed rounded end 2210 are further trimmed so that they are aligned with a proximal surface of the first distending member 104. Additionally, the inner and outer layers 308, 310 are sealed together at the junction between the first distending member 104 and the tubular connector 108, and between the tubular connector 108 and the second distending member 106, thereby forming the annular seals 110, 110′, respectively.

While embodiments and applications of the invention have been illustrated and described, it will be apparent to those skilled in the art that various modifications are possible without departing from the scope of the invention. It is, therefore, to be understood that within the scope of the appended claims, this invention may be practiced otherwise than as specifically described.

What is claimed is:

1. An expandable device for enlarging a body cavity, the device in its expanded configuration comprising:
first and second supporting members;
a tubular connector having inner and outer surfaces, the connector interconnecting the supporting members, the connector having a first end adjacent the first supporting member and a second end adjacent the second supporting member, the tubular connector having a maximum transverse dimension at its first end less than that of the first supporting member and a maximum transverse dimension at its second end less than that of the second supporting member, and the tubular connector having a length greater than the maximum transverse dimension of either the first supporting member or the second supporting member; and
a lumen defined by the inner surface of the tubular connector extending through the tubular connector;
wherein the tubular connector is adapted to apply force to the body cavity and retract surrounding tissue when the device is in the expanded configuration.

2. The device of claim 1, wherein each of the supporting members has an inflation chamber therein.

3. The device of claim 2, wherein the chambers of the first and second supporting members are separately inflatable.

4. The device of claim 1, wherein at least a substantial portion of the inner and outer surfaces of the tubular connector are not separatable.

5. The device of claim 4, wherein the tubular connector comprises an inner wall having the inner surface and an outer wall having the outer surface, the inner and outer walls connected together over a substantial portion of the connector.

6. The device of claim 5, wherein the inner and outer walls are connected at discrete locations so as to provide interconnected fluid paths.

7. The device of claim 6, wherein the interconnected fluid paths are in fluid communication with at least one inflation chamber provided in one of the first and second supporting members.

8. The device of claim 7, wherein the interconnected fluid paths are fluidly sealed from an inflation chamber provided in the second supporting member.

9. The device of claim 1, further comprising a plurality of rods between the inner and outer surfaces of the tubular connector.

10. The device of claim 9, wherein the rods are positioned between the inner and outer surfaces.

11. The device of claim 1, wherein the supporting members and the tubular connector are of a single piece construction composed of a single material.

12. The device of claim 2, further comprising at least one inflation tube disposed between the inner and outer surfaces of the tubular connector.

13. The device of claim 12, further comprising two inflation tubes, wherein one of the inflation tubes is in fluid communication with the inflation chamber of the first supporting member, and the other of the inflation tubes is in fluid communication with the inflation chamber of the second supporting member.

14. The device of claim 1, further comprising at least one opening in a side of the tubular connector through the inner and outer surfaces.

15. The device of claim 1, further comprising an end surface adjacent the first supporting member substantially closing the lumen adjacent the first end of the tubular connector.

16. The device of claim 15, further comprising at least one valve affixed to the end surface.

17. The device of claim 16, further comprising a duct in fluid communication with the central lumen.

18. The device of claim 1, wherein the tubular connector is substantially circular having substantially the same diameter at its first end and its second end.

19. The device of claim 1, wherein the tubular connector is substantially circular having a larger diameter at its first end than at its second end.

20. The device of claim 1, further comprising a third supporting member and a second tubular connector interconnecting the third supporting member and the second supporting member.

21. The device of claim 20, wherein at least one of the tubular connectors is cone-shaped.

22. The device of claim 1, wherein the tubular connector comprises a plurality of intermediate supporting members.

23. The device of claim 1, further comprising a plurality of lumens within the tubular connector.

24. The device of claim 1, wherein at least one of the first and second supporting members folds over a portion of the tubular connector.

25. The device of claim 1, further comprising a light source coupled to the device.

26. A device for enlarging a body cavity, the device comprising:
an elongate body having inner and outer surfaces extending between a first end of the elongate body and a second end of the elongate body, wherein a longitudinal dimension is generally defined between the first end and the second end with a transverse dimension being perpendicular to the longitudinal dimension;
a lumen defined by the inner surface of the elongate body extending through the elongate body;
a first supporting member connected adjacent the first end of the elongate body, the first supporting member having a maximum transverse dimension that is larger than a maximum transverse dimension of the elongate body at its first end;
a second supporting member connected adjacent the second end of the elongate body, the second supporting member having a maximum transverse dimension that is larger than a maximum transverse dimension of the elongate body at its second end;
wherein the elongate body has a length along its longitudinal dimension that is greater than the maximum transverse dimension of either the first supporting member or the second supporting member, and wherein the device is expandable between an undeployed position and a deployed position in which the outer surface of the elongate body exerts a force against a wall of the body cavity; and
an elongate applicator which retains the device for insertion into a body cavity, the device arranged on the applicator such that upon deployment the applicator is disposed in the lumen for withdrawal by a user.

27. The device of claim 26, wherein at least a portion of the outer surface of the elongate body has a generally circular cross-section.

28. The device of claim 26, wherein at least a portion of the outer surface of the elongate body has a generally triangular cross-section.

29. The device of claim 26, wherein at least a portion of the outer surface of the elongate body has a generally diamond-shaped cross-section.

30. The device of claim 26, wherein the first and second supporting members are expandable between an undeployed position and a deployed position.

31. The device of claim 30, wherein the first and second supporting members each include an inflation chamber.

32. The device of claim 30, wherein the elongate body is expandable between an undeployed position and a deployed position.

33. The device of claim 32, wherein the first and second supporting members are solid members.

34. The device of claim 32, comprising an inflation chamber between the inner and outer surfaces of the elongate body.

35. The device of claim 34, wherein at least a substantial portion of the inner and outer surfaces of the elongate body are not separatable.

36. The device of claim 35, wherein the elongate body includes a plurality of depressions defined by portions of the inner and outer surfaces connected together.

37. The device of claim 26, further comprising a plurality of rods between the inner and outer surfaces of the elongate body.

38. The device of claim 26, wherein the first and second supporting members and the outer surface of the elongate body have a generally circular cross-section.

39. The device of claim 38, wherein the outer surface of the elongate body has substantially the same diameter at the first end and the second end.

40. The device of claim 38, wherein the outer surface of the elongate body has a larger diameter at the first end than at the second end.

41. The device of claim 26, wherein at least one of the first and second supporting members has a generally triangular cross-section.

42. The device of claim 26, wherein at least one of the first and second supporting members has generally diamond-shaped cross-section.

43. A method of examining a body cavity, the method comprising:
inserting an expandable device into the body cavity, the expandable device having a proximal end and a distal end and an inner and outer surface extending between the proximal and distal ends, and a lumen defined by the inner surface extending between the proximal end and the distal end, wherein the longitudinal length between the proximal and distal ends is greater than the maximum transverse dimension of either of the proximal and distal ends, and the outer surface between the proximal and distal ends has a maximum transverse dimension that is less than the maximum transverse dimension of either of the proximal and distal ends; and
expanding the expandable device within the body cavity, wherein expansion of the expandable device causes the outer surface between the proximal and distal ends to exert a force against a wall of the body cavity.

44. The method of claim 43, wherein expanding the expandable device comprises inflating at least one inflation chamber provided within the expandable device.

45. The method of claim 43, wherein the proximal and distal ends of the expandable device each includes a supporting member.

46. The method of claim 45, wherein the supporting members at each of the proximal and distal ends are expandable.

47. The method of claim 46, comprising inflating the expandable supporting members with a fluid.

48. The method of claim 47, wherein expanding the expandable device comprises separately inflating each of the supporting members.

49. The method of claim 45, wherein expanding the expandable device comprises expanding a connection region extending between the supporting members.

50. The method of claim 49, wherein expanding the connecting region comprises inflating a chamber provided between the inner and outer surfaces.

51. The method of claim 50, expanding the expandable device further comprises inflating a chamber provided within each of the supporting members.

52. The method of claim 51, wherein the chamber of the supporting member at the proximal end of the device and the chamber of the connecting region are in fluid communication.

53. The method of claim 52, wherein the chambers of the supporting member at the proximal end of the device and the connecting region are inflated separately from the chamber of the supporting member at the distal end of the device.

54. The method of claim 43, further comprising delivering at least one medical instrument through the lumen.

55. The method of claim 43, further comprising performing visualization through the lumen.

56. The method of claim 43, further comprising deactuating the expandable device to a contracted configuration.

57. The method of claim 56, wherein deactuating the expandable device comprises contracting at least the proximal end of the device prior to contracting the distal end of the device.

58. The method of claim 43, wherein the body cavity is the vagina.

59. The method of claim 43, wherein the body cavity is the cervix.

60. An apparatus comprising an expandable device having a lumen and an applicator for inserting the expandable device into a body cavity, the applicator comprising:
a retaining portion which holds at least a portion of the expandable device in a collapsed state while the expandable device is inserted into the body cavity;
a handle portion; and
a shaft portion extending through the lumen between the retaining portion and the handle portion.

61. The apparatus of claim 60, wherein the expandable device is inflatable.

62. The apparatus of claim 60, wherein the applicator is of a one-piece design.

63. The apparatus of claim 60, wherein the retaining portion comprises a curved portion formed at a distal end of the applicator.

64. The apparatus of claim 60, wherein the retaining portion comprises a retaining bell connected to a distal end of the shaft portion for receiving a distal end of the expandable device.

65. The apparatus of claim 64, further comprising a second retaining bell connected along an intermediate portion of the shaft portion receiving a proximal end of the expandable device.

66. The apparatus of claim 65, wherein the second retaining bell is slidable relative to the shaft portion.

67. The apparatus of claim 60, wherein the retaining portion includes a sleeve having a retaining cavity and a tear-line.

68. A method of inserting an expandable device into a body cavity, the expandable device having a proximal end and a distal end and a lumen extending therethrough, the method comprising:
inserting the expandable device and the applicator into a desired position with the body cavity, the expandable device being at least partially retained within a retaining portion of the applicator;
expanding the expandable device; and
withdrawing the applicator through the lumen of the expandable device.

69. The method of claim 68, wherein the expandable device is an inflatable device.

70. The method of claim 68, wherein the retaining portion comprises a curved portion formed at a distal end of the shaft portion.

71. The method of claim 68, wherein the retaining portion comprises a retaining bell connected to a distal end of the shaft portion.

72. The method of claim 68, wherein the retaining portion includes a finger cot having a retaining cavity and a tear-line.
